SEQUENCE LISTING (99 sequences)

SEQ ID NO: 1 - protein, 133 aa, synthetic construct
APTSSSTKKTQLQLEHLLLALQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, synthetic construct
APTSSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 133 aa, synthetic construct
APTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 133 aa, synthetic construct
APTSSSTKKTQLQLEHLLLDLQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 5 - protein, 133 aa, synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 133 aa, synthetic construct
APTSSSTKKTQLQLEHLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 133 aa, synthetic construct
APTSSSTKKTQLQLEHLLLILQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 133 aa, synthetic construct
APTSSSTKKTQLQLEHLLLSLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 133 aa, synthetic construct
APTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, synthetic construct
APTSSSTKKTQLQLEHLLLALQMILNGINNYKNPKLTNMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 11 - protein, 133 aa, synthetic construct
APTSSSTKKTQLQLEHLLLALQMILNGINNYKNPKLTGMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 133 aa, synthetic construct
APTSSSTKKTQLQLEHLLLALQMILNGINNYKNPKLTHMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 13 - protein, 133 aa, synthetic construct
APTSSSTKKTQLQLEHLLLALQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 14 - protein, 133 aa, synthetic construct
APTSSSTKKTQLQLEHLLLALQMILNGINNYKNPKLTLMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 15 - protein, 133 aa, synthetic construct
APTSSSTKKTQLQLEHLLLALQMILNGINNYKNPKLTMMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 16 - protein, 133 aa, synthetic construct
APTSSSTKKTQLQLEHLLLALQMILNGINNYKNPKLTFMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 17 - protein, 133 aa, synthetic construct
APTSSSTKKTQLQLEHLLLALQMILNGINNYKNPKLTPMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 18 - protein, 133 aa, synthetic construct
APTSSSTKKTQLQLEHLLLALQMILNGINNYKNPKLTSMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 19 - protein, 133 aa, synthetic construct
APTSSSTKKTQLQLEHLLLALQMILNGINNYKNPKLTTMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 20 - protein, 133 aa, synthetic construct
APTSSSTKKTQLQLEHLLLALQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 21 - protein, 133 aa, synthetic construct
APTSSSTKKTQLQLEHLLLALQMILNGINNYKNPKLTYMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 22 - protein, 133 aa, synthetic construct
APTSSSTKKTQLQLEHLLLALQMILNGINNYKNPKLTVMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 133 aa, synthetic construct
APTSSSTKKTQLQLEHLLLALQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 24 - protein, 133 aa, synthetic construct
APTSSSTKKTQLQLEHLLLALQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 25 - protein, 133 aa, synthetic construct
APTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 26 - protein, 133 aa, synthetic construct
APTSSSTKKTQLQLEHLLLALQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - protein, 133 aa, synthetic construct
APTSSSTKKTQLQLEHLLLNLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 28 - protein, 133 aa, synthetic construct
APTSSSTKKTQLQLEHLLLQLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 29 - protein, 133 aa, synthetic construct
APTSSSTKKTQLQLEHLLLELQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 30 - protein, 133 aa, synthetic construct
APTSSSTKKTQLQLEHLLLGLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 31 - protein, 133 aa, synthetic construct
APTSSSTKKTQLQLEHLLLILQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 32 - protein, 133 aa, synthetic construct
APTSSSTKKTQLQLEHLLLMLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 33 - protein, 133 aa, synthetic construct
APTSSSTKKTQLQLEHLLLTLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 34 - protein, 133 aa, synthetic construct
APTSSSTKKTQLQLEHLLLVLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 35 - protein, 133 aa, synthetic construct
APTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 36 - protein, 133 aa, synthetic construct
APASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 37 - protein, 133 aa, synthetic construct
APASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 38 - protein, 130 aa, synthetic construct
SSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 39 - protein, 130 aa, synthetic construct
SSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 40 - protein, 133 aa, synthetic construct
APTSSSTKKTQLQLEHLLLSLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 41 - protein, 133 aa, synthetic construct
APTSSSTKKTQLQLEHLLLALQMILNGINNYKNPKLTKMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 42 - protein, 133 aa, synthetic construct [sequence omitted]

SEQ ID NO: 43 - protein, 122 aa, synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSS

SEQ ID NO: 44 - protein, 109 aa, synthetic construct
EIVLTQSPGTLSLSPGERATLSCRASQSIGKSFLAWYQQKPGQAPRLLIYDASTRAADIPARFSGSGSGTDFTLTISSLEPEDFAVYYCQQYYDWPPLSFGGGTKVEIK

SEQ ID NO: 45 - protein, 10 aa, synthetic construct
GFTFKDYCMT

SEQ ID NO: 46 - protein, 17 aa, synthetic construct
AIVYSGGSTYYADSVKG

SEQ ID NO: 47 - protein, 13 aa, synthetic construct
YTRASYFYDAMDV

SEQ ID NO: 48 - protein, 12 aa, synthetic construct
RASQSIGKSFLA

SEQ ID NO: 49 - protein, 7 aa, synthetic construct
DASTRAA

SEQ ID NO: 50 - protein, 10 aa, synthetic construct
QQYYDWPPLS

SEQ ID NO: 51 - protein, 121 aa, synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSS

SEQ ID NO: 52 - protein, 108 aa, synthetic construct
EIVLTQSPDFQSVTPKEKVTITCTASESVPPQFLHWYQQKPDQSPKLLIYASRERASGVPSRFSGSGSGTDFTLTINSLEAEDAATYYCHQFHRSPLTFGGGTKLEIK

SEQ ID NO: 53 - protein, 10 aa, synthetic construct
GYSLYGTSMH

SEQ ID NO: 54 - protein, 17 aa, synthetic construct
YISPFTGRATYAQGFTG

SEQ ID NO: 55 - protein, 12 aa, synthetic construct
DYDYRYYYAMDY

SEQ ID NO: 56 - protein, 12 aa, synthetic construct
TASESVPPQFLH

SEQ ID NO: 57 - protein, 7 aa, synthetic construct
ASRERAS

SEQ ID NO: 58 - protein, 9 aa, synthetic construct
HQFHRSPLT

SEQ ID NO: 59 - protein, 115 aa, synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSS

SEQ ID NO: 60 - protein, 110 aa, synthetic construct
QSVLTQPPSASGTPGQRVTISCSGSTSNIGRESVYWYQQLPGTAPKLLIYSNVQRPSGAPNRFSGSKSGTSASLAISGLQSEDEADYYCGTWDDSLNGWVFGGGTKLTVL

SEQ ID NO: 61 - protein, 10 aa, synthetic construct
GFTFDISAMS

SEQ ID NO: 62 - protein, 17 aa, synthetic construct
TISGSAYSTYYADSVKG

SEQ ID NO: 63 - protein, 6 aa, synthetic construct
EIFSDY

SEQ ID NO: 64 - protein, 13 aa, synthetic construct
SGSTSNIGRESVY

SEQ ID NO: 65 - protein, 7 aa, synthetic construct
SNVQRPS

SEQ ID NO: 66 - protein, 11 aa, synthetic construct
GTWDDSLNGWV

SEQ ID NO: 67 - protein, 451 aa, synthetic construct
EVQLLESGGGLVQPGGSLRLSCAASGFTFKSYAMHWVRQAPGKGLEWVSAIVYSGGSTYYADSVKGRFTISRDNSKNTLYLQMNSLRAEDTAVYYCAKYDRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 216 aa, synthetic construct
EIVLTQSPGTLSLSPGERATLSCRASQSISSSFLAWYQQKPGQAPRLLIYDASDRATGIPDRFSGSGSGTDFTLTISRLEPEDFAVYYCQQYYDWPPLSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 122 aa, synthetic construct
EVQLLESGGGLVQPGGSLRLSCAASGFTFKSYAMHWVRQAPGKGLEWVSAIVYSGGSTYYADSVKGRFTISRDNSKNTLYLQMNSLRAEDTAVYYCAKYDRASYFYDAMDVWGQGTTVTVSS

SEQ ID NO: 70 - protein, 109 aa, synthetic construct
EIVLTQSPGTLSLSPGERATLSCRASQSISSSFLAWYQQKPGQAPRLLIYDASDRATGIPDRFSGSGSGTDFTLTISRLEPEDFAVYYCQQYYDWPPLSFGGGTKVEIK

SEQ ID NO: 71 - protein, 10 aa, synthetic construct
GFTFKSYAMH

SEQ ID NO: 72 - protein, 17 aa, synthetic construct
AIVYSGGSTYYADSVKG

SEQ ID NO: 73 - protein, 13 aa, synthetic construct
YDRASYFYDAMDV

SEQ ID NO: 74 - protein, 12 aa, synthetic construct
RASQSISSSFLA

SEQ ID NO: 75 - protein, 7 aa, synthetic construct
DASDRAT

SEQ ID NO: 76 - protein, 10 aa, synthetic construct
QQYYDWPPLS

SEQ ID NO: 77 - protein, 448 aa, synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 78 - protein, 216 aa, synthetic construct
EIVLTQSPGTLSLSPGERATLSCRASQSIGKSFLAWYQQKPGQAPRLLIYDASTRAADIPARFSGSGSGTDFTLTISSLEPEDFAVYYCQQYYDWPPLSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 447 aa, synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 215 aa, synthetic construct
EIVLTQSPDFQSVTPKEKVTITCTASESVPPQFLHWYQQKPDQSPKLLIYASRERASGVPSRFSGSGSGTDFTLTINSLEAEDAATYYCHQFHRSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 441 aa, synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - protein, 216 aa, synthetic construct
QSVLTQPPSASGTPGQRVTISCSGSTSNIGRESVYWYQQLPGTAPKLLIYSNVQRPSGAPNRFSGSKSGTSASLAISGLQSEDEADYYCGTWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83 - protein, 585 aa, synthetic construct
EVQLLESGGGLVQPGGSLRLSCAASGFTFKSYAMHWVRQAPGKGLEWVSAIVYSGGSTYYADSVKGRFTISRDNSKNTLYLQMNSLRAEDTAVYYCAKYDRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 84 - protein, 367 aa, synthetic construct
GAGGTGCAGCTGCTGGAAAGCGGCGGCGGACTGGTGCAGCCTGGAGGCAGCCTGCGGCTGTCTTGTGCCGCTTCTGGCTTCACCTTCAAGGACTACTGCATGACCTGGGTCAGACAGGCCCCTGGCAAGGGCCTCGAGTGGGTGTCCGCCATCGTGTACAGCGGCGGGTCAACATACTACGCCGACAGCGTGAAGGGCAGATTCACAATCAGCAGAGATAACAGCAAGAACACCCTGTACCTGCAGATGAACAACCTGAGAGCTGAAGATACCGCCGTGTACTACTGCGCCAAGTACACCAGAGCCAGCTACTTCTACGACGCCATGGACGTGTGGGGCCAGGGCACCACCGTGACAGTGTCCTCAT

SEQ ID NO: 85 - protein, 327 aa, synthetic construct
GAGATCGTGCTGACCCAGTCTCCTGGCACCCTGAGCCTGAGCCCTGGCGAGAGAGCTACACTGTCATGCAGAGCCTCTCAGAGCATCGGCAAGAGCTTCCTGGCCTGGTACCAGCAAAAGCCTGGACAGGCCCCTAGACTGCTGATCTACGACGCCAGCACCAGAGCCGCTGATATCCCCGCCAGATTCAGCGGATCTGGCAGCGGCACTGATTTCACCCTCACCATCAGCAGCCTGGAACCCGAGGACTTCGCCGTGTACTACTGCCAGCAGTACTACGACTGGCCCCCCCTGTCTTTTGGCGGAGGCACAAAGGTGGAAATCAAG

SEQ ID NO: 86 - protein, 363 aa, synthetic construct
CAGGTTCAGCTGGTTCAGTCTGGCAGCGAGCTGAAGAAACCTGGCGCCTCTGTGAAGGTGTCCTGCAAGGCCTCTGGCTACAGCCTGTACGGCACCTCTATGCACTGGGTCCGACAGGCTCCAGGACAGGGACTTGAGTGGATGGGCTACATCAGCCCCTTTACCGGCAGAGCCACATACGCCCAGGGCTTCACAGGCAGATTCGTGTTCAGCCTGGACACCAGCGTGTCCACAGCCTACCTGCAGATCAGCTCTCTGAAGGCCGAGGACACCGCCGTGTACTACTGCGCCAGAGACTACGACTACCGGTACTACTATGCCATGGACTACTGGGGCCAGGGCACCACAGTTACAGTGTCCTCA

SEQ ID NO: 87 - protein, 324 aa, synthetic construct
GAAATTGTGCTGACACAGAGCCCCGACTTCCAGAGCGTGACCCCTAAAGAAAAAGTGACCATCACCTGTACCGCCAGCGAGTCCGTGCCTCCTCAGTTCCTGCATTGGTATCAGCAGAAGCCCGATCAGAGCCCCAAGCTGCTGATCTACGCCAGCAGAGAAAGAGCCAGCGGCGTCCCAAGCAGATTTTCTGGCTCTGGCAGCGGCACCGACTTCACCCTGACAATCAATAGCCTGGAAGCCGAGGACGCCGCCACCTACTACTGCCACCAGTTTCACAGAAGCCCTCTGACCTTTGGCGGAGGCACCAAGCTGGAAATCAAG

SEQ ID NO: 88 - protein, 345 aa, synthetic construct
GACGTGCAGCTGGTGGAAAGCGGCGGAGGCCTGGTCCAGCCCGGCGGCTCTCTGAGACTGAGCTGCGCCGCCAGCGGCTTCACCTTCGACATCAGCGCCATGAGCTGGGTGCGGCAGGCCCCTGGCAAGGGCCTGGAATGGGTCAGCACAATCAGCGGATCTGCCTACAGCACCTACTACGCCGACAGCGTGAAGGGCAGATTCACCATCTCAAGAGATAACAGCAAGAGCACCCTGTACCTGCAGATGAACAGCCTGCGGGCCGAGGACACCGCCGTGTACTACTGCGCCAGAGAGATCTTCAGCGACTACTGGGGCTTGGGCACCCTGGTGACAGTGTCCTCA

SEQ ID NO: 89 - protein, 330 aa, synthetic construct
CAAAGCGTGCTGACACAGCCCCCCAGCGCTTCTGGCACCCCTGGCCAGAGAGTGACCATCTCATGCAGCGGGTCAACAAGCAACATCGGCAGAGAGAGCGTGTACTGGTACCAGCAGCTGCCTGGAACCGCCCCTAAGCTGCTGATCTACAGCAACGTGCAGCGGCCTAGCGGCGCCCCTAACAGATTCAGCGGCAGCAAGAGCGGCACCAGCGCCAGCCTGGCCATCAGCGGCCTGCAGAGCGAGGACGAGGCCGACTACTACTGCGGCACATGGGACGACAGCCTGAACGGCTGGGTGTTCGGCGGCGGAACTAAGCTGACCGTCCTA

SEQ ID NO: 90 - protein, 582 aa, synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 91 - protein, 575 aa, synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 92 - protein, 582 aa, synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 93 - protein, 585 aa, synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 94 - protein, 582 aa, synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 95 - protein, 575 aa, synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLSLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 96 - protein, 581 aa, synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 97 - protein, 581 aa, synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 98 - protein, 585 aa, synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 99 - protein, 585 aa, synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLSLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

(19) United States
(12) Patent Application Publication
(10) Pub. No.: US 2026/0083857 A1
(43) Pub. Date: Mar. 26, 2026

(54) ANTI-PD-1 ANTIBODY-ATTENUATED IL-2 IMMUNOCONJUGATE FORMULATIONS

(71) Applicant: Cephalon LLC, West Chester, PA (US)

(72) Inventors: Haichen Nie, Exton, PA (US); Pragya Manandhar, Media, PA (US); Zaneta Maijorovaite, Phoenixville, PA (US); Wei Liu, Exton, PA (US)

(21) Appl. No.: 19/335,771

(22) Filed: Sep. 22, 2025

Related U.S. Application Data
(60) Provisional application No. 63/697,796, filed on Sep. 23, 2024.

(51) Int. Cl. A61K 47/68; A61K 47/12; A61K 47/18; A61K 47/26; C07K 14/55; C07K 16/28

(52) U.S. Cl. CPC A61K 47/6851; A61K 47/12; A61K 47/183; A61K 47/26; A61K 47/6813; C07K 14/55; C07K 16/2818; C07K 2317/92

(57) ABSTRACT

Disclosed herein are stable pharmaceutical formulations comprising anti-human PD-1 (hPD-1) antibody-modified human interleukin-2 (hIL-2) immunoconjugates and uses thereof.

### CROSS REFERENCE TO RELATED APPLICATIONS

This application claims priority to U.S. Provisional Application No. 63/697,796, which was filed on Sep. 23, 2024, the disclosure of which is hereby incorporated by reference in its entirety.

### SEQUENCE LISTING

The instant application contains a Sequence Listing which is being submitted herewith electronically in XML format and is hereby incorporated by reference in its entirety. Said XML copy, created on Sep. 16, 2025, is named BIO069-WO01_102085002651_SequenceListing_ST26.xml and is 1,03,897 bytes in size.

### TECHNICAL FIELD

Disclosed herein are stable pharmaceutical formulations comprising anti-human PD-1 (hPD-1) antibody-modified human interleukin-2 (hIL-2) immunoconjugates and uses thereof.

### BACKGROUND

The past decade has seen dramatic benefits from immuno-oncology therapies for the treatment of cancer. The approval of immunotherapies, such as immune checkpoint inhibitors, adoptive cell therapies and cancer vaccines, having revolutionized the way cancer treatment is approached. These developments have altered the standard of care (SOC) and improved survival for several tumor types. However, while immune checkpoint inhibitors have improved clinical outcomes in a variety of tumor types, only a subset of patients show clinical responses, and a large group of responders develop acquired resistance after an initial response.

The efficacy of immune checkpoint inhibitors such as PD-1 antagonists requires patients to have a competent immune system and adequate immune cell numbers. Non-responders to PD-1 antagonists typically exhibit low tumor T-cell infiltration and poor proliferative T-cell responses to PD-1 antagonism.

Human IL-2 (hIL-2) is a Type 1 four α-helical bundle, glycosylated cytokine produced by CD4+ T cells and CD8+ T cells. Autocrine and paracrine IL-2 signaling occurs through engagement of either a high-affinity trimeric receptor complex comprising IL-2Rα (CD25), IL-2Rβ (CD122), and IL-2Rγ (CD132), or an intermediate-affinity dimeric receptor complex which comprises IL-2Rβ (CD122) and IL-2Rγ (CD132). IL-2 has dual opposing and pleiotropic roles, in that it can both stimulate T cell proliferation to generate T cell effector, T cell memory, and activated NK cells, but can also stimulate suppressive regulatory T cells for maintenance of immune homeostasis. Low-dose IL-2 primarily stimulates regulatory T cells as well as some T effector and NK cells, whereas high-dose IL-2 broadly stimulates cytotoxic T cells, T effector, and NK cells and regulatory T cells. The use of IL-2 in the treatment of autoimmune diseases and as a cancer immunotherapy has, however, been limited by off-target effects and toxicity associated with the administration of IL-2.

### SUMMARY

Formulation of an antibody-IL2 immunoconjugate can be challenging because the optimal conditions for a stable soluble antibody solution may not be optimal for the IL-2 species, and vice versa. Further complications may be added by the addition of various attenuating mutations in IL-2. We have identified a range of conditions optimal for a stable solution of the anti-human PD-1 (hPD-1) antibody-modified human interleukin-2 (hIL-2) immunoconjugate comprising (a) a modified hIL-2 protein comprising the amino acid sequence of SEQ ID NO: 25; and an anti-hPD-1 antibody, or antigen-binding fragment thereof, that immunospecifically binds to hPD-1, wherein the antibody or antigen-binding fragment thereof, comprises a heavy chain complementarity determining region 1 (CDR1) comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.

Disclosed herein are stable pharmaceutical formulations comprising:
about 18 mM to about 22 mM of acetate;
about 50 mM to about 250 mM of sucrose;
about 100 mM to about 500 mM of glycine;
about 0.05% to about 0.1% of PS80; and
about 5 mg/mL to about 10 mg/mL of an anti-human PD-1 (hPD-1) antibody-modified human interleukin-2 (hIL-2) immunoconjugate comprising:
   a modified hIL-2 protein comprising the amino acid sequence of SEQ ID NO: 25; and
   an anti-hPD-1 antibody, or antigen-binding fragment thereof, that immunospecifically binds to hPD-1, wherein the antibody or antigen-binding fragment thereof, comprises a heavy chain complementarity determining region 1 (CDR1) comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.

The acetate in the stable pharmaceutical formulation may be sodium acetate. The formulation may be buffered to about pH 4.5-5.5.

Disclosed herein are stable lyophilized pharmaceutical formulations prepared by lyophilizing any of the disclosed stable pharmaceutical formulations.

Disclosed herein are methods of treating a cancer in a subject, the methods comprising administering a therapeutically effective amount of any of the disclosed stable pharmaceutical formulations to the subject to thereby treat the cancer.

### BRIEF DESCRIPTION OF THE DRAWINGS

The summary, as well as the following detailed description, is further understood when read in conjunction with the appended drawings. For the purpose of illustrating the disclosed formulations and methods, there are shown in the drawings exemplary embodiments of the formulations and methods; however, the formulations and methods are not limited to the specific embodiments disclosed. In the drawings:

FIG. 1A, FIG. 1B, FIG. 1C, FIG. 1D, FIG. 1E, FIG. 1F, FIG. 1G, FIG. 1H, and FIG. 1I, illustrate the results from the buffer and pH screening study.

FIG. 2A, FIG. 2B, FIG. 2C, and FIG. 2D illustrate the results from the surfactant screening study and the addition of stabilizer.

FIG. 3 illustrates the sub-visible particle counts for the protein-protein modifier screening study.

FIG. 4A, FIG. 4B, FIG. 4C, and FIG. 4D illustrate the results from the evaluation of formulations at different protein concentrations.

FIG. 5A, FIG. 5B, and FIG. 5C illustrate the results from the evaluation of formulations at different protein concentrations.

FIG. 6 illustrates the results from studies evaluating different amino acids at varying concentrations.

FIG. 7A, FIG. 7B, FIG. 7C, FIG. 7D, FIG. 7E, and FIG. 7F illustrate the results from glycine and sucrose concentration range studies.

FIG. 8 illustrates the thermal stability profile of H7-767 (also referred to herein as H7-632-IgG1-LAGA-IL-2AAEA) in different buffer and pH conditions.

In the figures: TO refers to the initial time point; 3 FT refers to 3 rounds of Freeze/Thaw; Tₒₙₛₑₜ refers to the onset temperature at which the anti-hPD-1 antibody-modified hIL-2 immunoconjugate begins to unfold/denature; Tₘ refers to the temperature mid-point at which the anti-hPD-1 antibody-modified hIL-2 immunoconjugate unfolds (i.e., the temperature at which half the anti-hPD-1 antibody-modified hIL-2 immunoconjugate molecules are folded and half are unfolded); and 1 month at 40° C. refers to the stress conditions of 40° C. 75% relative humidity (RH) for 1 month.

### DETAILED DESCRIPTION OF ILLUSTRATIVE EMBODIMENTS

The disclosed formulations and methods may be understood more readily by reference to the following detailed description taken in connection with the accompanying figures, which form a part of this disclosure.

Unless specifically stated otherwise, any description as to a possible mechanism or mode of action or reason for improvement is meant to be illustrative only, and the disclosed formulations and methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the formulations and methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise. The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

It is to be appreciated that certain features of the disclosed formulations and methods which are, for clarity, described herein in the context of separate embodiments, may also be provided in combination in a single embodiment. Conversely, various features of the disclosed formulations and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of;” similarly, the term “consisting essentially of” is intended to include examples encompassed by the term “consisting of.”

For the excipients used in the formulation the following definitions may be applied.

“Acetate” may encompass any non-toxic acetate, irrespective of the counter ion. The claimed concentration of the acetate is determined by the acetate species. Most typically, acetate refers to sodium acetate.

PS80 refers to polysorbate 80, also known as polyoxyethylene (20) sorbitan monooleate.

“Glycine” and “Sucrose” are terms well known in the art. Is may be possible to replace sucrose with trehalose and/or mannitol.

The term “antibody” is meant in a broad sense and includes full length immunoglobulin molecules and antigen-binding fragments thereof.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antigen-binding fragment” refers to a portion of an immunoglobulin molecule that retains the antigen binding properties of the parental full length antibody (i.e., “antigen-binding fragment thereof”). Exemplary antigen binding fragments can have: heavy chain complementarity determining regions (CDR) 1, 2, and/or 3; light chain CDR 1, 2, and/or 3; a heavy chain variable region (VH); a light chain variable region (VL); and combinations thereof. Antigen binding fragments include: a Fab fragment (a monovalent fragment consisting of the VL, VH, constant light (CL), and constant heavy 1 (CH1) domains); a F (ab) 2 fragment (a bivalent fragment comprising two Fab fragments linked by a disulfide bridge at the hinge region); a Fd fragment consisting of the VH and CH1 domains; a Fv fragment consisting of the VL and VH domains of a single arm of an antibody; and a domain antibody (dAb) fragment (Ward et al., Nature 341:544-546, 1989), which consists of a VH domain or a VL domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody, described for example in Int'l Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

The phrase “immunospecifically binds” refers to the ability of the disclosed antibodies or antigen-binding fragments thereof to preferentially bind to its target (hPD-1) without preferentially binding other molecules in a sample containing a mixed population of molecules. Antibodies that immunospecifically bind hPD-1 are substantially free of other antibodies having different antigenic specificities (e.g., an anti-hPD-1 antibody is substantially free of antibodies that specifically bind antigens other than hPD-1). Antibodies that immunospecifically bind hPD-1, however, can have cross-reactivity to other antigens, such as orthologs of hPD-1, including Macaca fascicularis (cynomolgus monkey) PD-1. The antibodies disclosed herein are able to immunospecifically bind both naturally-produced hPD-1 and to PD-1 which is recombinantly produced in mammalian or prokaryotic cells.

An antibody variable region consists of four “framework” regions interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132:211-50, 1970; Kabat et al., Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196:901-17, 1987). The AbM definition of CDRs is also widely used; it is a compromise between Kabat and Chothia numbering schemes and is so-called because it was used by Oxford Molecular's AbM antibody modelling software (Rees, A. R., Searle, S. M. J., Henry, A. H. and Pedersen, J. T. (1996) In Sternberg M. J. E. (ed.), Protein Structure Prediction. Oxford University Press, Oxford, 141-172). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Treat,” “treatment,” and like terms includes reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by the cancer. Treatment also includes prolonging survival as compared to the expected survival of a subject not receiving treatment.

Throughout this text, the descriptions refer to formulations and methods. Where the disclosure describes or claims a feature or embodiment associated with a formulation, such a feature or embodiment is equally applicable to the use of that formulation. Likewise, where the disclosure describes or claims a feature or embodiment associated with the use of a formulation, such a feature or embodiment is equally applicable to the formulation. Uses included within the scope of this disclosure include, but are not limited to, Swiss-style uses, first medical uses, second/further medical uses, and uses in medicine.

As used herein, “administering” and similar terms indicate a procedure by which the disclosed stable pharmaceutical formulations are introduced into a subject such that target cells, tissues, or segments of the body of the subject are contacted with the disclosed formulations (specifically the anti-hPD-1 antibody-modified hIL-2 immunoconjugate). Typically, the formulation is added first to an isotonic fluid for IV administration. Isotonic fluids are well known, and include saline, dextrose, Ringers solution, and the like. The isotonic fluid may also contain other therapeutic agents. The pharmaceutical formulation may be administered into a subject via intravenous (IV) infusion.

The phrase “therapeutically effective amount” refers to an amount of the stable pharmaceutical formulations, as described herein, effective to achieve a particular biological or therapeutic result such as, but not limited to, biological or therapeutic results disclosed, described, or exemplified herein. The therapeutically effective amount may vary according to factors such as the disease state, age, sex, and weight of the individual, and the ability of the immunoconjugate to cause a desired response in a subject. Exemplary indicators of a therapeutically effect amount include, for example, improved well-being of the patient, reduction of a cancer symptom, arrested or slowed progression of cancer symptoms, and/or absence of cancer symptoms.

The term “subject” as used herein is intended to mean any mammal, and preferably a human or non-human primate. “Subject” and “patient” are used interchangeably herein.

The disclosed modified hIL-2 proteins are also referred to as “attenuated” hIL-2 herein. As described herein the term “reduced potency” and related terms such as “reduction in potency” or “attenuation” of IL-2 activity refer to a reduction in potency of the modified hIL-2 as determined by an increased EC₅₀ value relative to the EC₅₀ value for an non-modified-hIL-2 in an IL-2-dependent assay. As described herein the reduction in potency of the modified hIL-2 will be on both the high affinity and on the intermediate affinity IL-2 receptors. The IL-2-dependent assay for determining potency may be an engineered human erythroleukemic TF1 (TF1+IL-2Rβ) or a human natural killer NK-92 cell proliferation assay as described herein. In one embodiment, the IL-2-dependent assay for determining potency is an engineered human erythroleukemic TF1 (TF1+IL-2Rβ) cell proliferation assay. In another embodiment, the IL-2-dependent assay for determining potency is a human natural killer NK-92 cell proliferation assay. Other IL-2-dependent assays for determining potency may also be a TF1+IL-2Rβ or a human natural killer NK-92 pSTAT5 assay as described herein. The non-modified-hIL-2 used in the IL-2-dependent assay may be a prokaryote-expressed hIL-2 such as Proleukin® (which has the native human IL-2 amino acid sequence apart from a C125S substitution to remove an unbound cysteine, and which does not bear the normal human carbohydrate expression on residue T3), or the non-modified-hIL-2 used in the IL-2-dependent assay may be an hIL-2 with the amino acid sequence of SEQ ID NO: 42 or with the amino acid sequence of SEQ ID NO: 42 with a C125S substitution, which is expressed in a mammalian cell line, such as a CHO or HEK cell line.

Immunoconjugate and fusion protein are used interchangeably herein.

Disclosed herein are stable pharmaceutical formulations comprising:
about 18 mM to about 22 mM of acetate;
about 50 mM to about 250 mM of sucrose;
about 100 mM to about 500 mM of glycine;
about 0.05% to about 0.1% of PS80; and
about 5 mg/mL to about 10 mg/mL of an anti-human PD-1 (hPD-1) antibody-modified human interleukin-2 (hIL-2) immunoconjugate.

Suitable anti-hPD-1 antibody-modified hIL-2 immunoconjugates include those disclosed in U.S. Pub. No 2024/0076343.

The anti-hPD-1 antibody-modified hIL-2 immunoconjugate can comprise a modified hIL-2 protein that comprises a substitution at amino acid position 20 relative to the non-modified hIL-2 amino acid sequence of SEQ ID NO: 42 and a substitution at amino acid position 38 relative to the non-modified hIL-2 amino acid sequence of SEQ ID NO: 42.

Suitable substitutions at amino acid position 20 of the modified hIL-2 portion of the immunoconjugates include, for example, any of a D20A, D20S, D20Q, D20M, D20I, D20V, D20N, D20G, D20T, or D20E substitution relative to the non-modified hIL-2 amino acid sequence of SEQ ID NO: 42.

Suitable substitutions at amino acid position 38 of the modified hIL-2 portion of the immunoconjugates include, for example, any of an R38E, R38N, R38G, R38H, R38I, R38L, R38M, R38F, R38P, R38S, R38T, R38W, R38Y, R38V, R38A, R38Q, R38D, or a R38K substitution relative to the non-modified hIL-2 amino acid sequence of SEQ ID NO: 42.

In some embodiments, any one of the D20A, D20S, D20Q, D20M, D20I, D20V, D20N, D20G, D20T, or D20E substitutions can be combined with an R38E substitution. Thus, the anti-hPD-1 antibody-modified hIL-2 immunoconjugate can comprise a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 42 and an R38E substitution at amino acid position 38 relative to the non-modified hIL-2 amino acid sequence of SEQ ID NO: 42. The anti-hPD-1 antibody-modified hIL-2 immunoconjugate may comprise a modified hIL-2 protein comprising the amino acid sequence of SEQ ID NO: 25. The modified hIL-2 portion of the immunoconjugate may comprise D20A and R38E substitutions relative to the non-modified hIL-2 amino acid sequence of SEQ ID NO: 42.

The modified hIL-2 protein portion of the immunoconjugates can comprise the amino acid sequence of any one of SEQ ID NOs: 10-34, 40, or 41. The modified hIL-2 protein portion of the immunoconjugates can comprise the amino acid sequence of any one of SEQ ID NOs: 10-26, 28, 31, 32, 34, 40, or 41. The modified hIL-2 protein portion of the immunoconjugates can comprise the amino acid sequence of any one of SEQ ID NOs: 25, 27-34, or 40. In some embodiments, the modified hIL-2 protein portion of the immunoconjugates comprise the amino acid sequence of SEQ ID NO: 10. In some embodiments, the modified hIL-2 protein portion of the immunoconjugates comprise the amino acid sequence of SEQ ID NO: 11. In some embodiments, the modified hIL-2 protein portion of the immunoconjugates comprise the amino acid sequence of SEQ ID NO: 12. In some embodiments, the modified hIL-2 protein portion of the immunoconjugates comprise the amino acid sequence of SEQ ID NO: 13. In some embodiments, the modified hIL-2 protein portion of the immunoconjugates comprise the amino acid sequence of SEQ ID NO: 14. In some embodiments, the modified hIL-2 protein portion of the immunoconjugates comprise the amino acid sequence of SEQ ID NO: 15. In some embodiments, the modified hIL-2 protein portion of the immunoconjugates comprise the amino acid sequence of SEQ ID NO: 16. In some embodiments, the modified hIL-2 protein portion of the immunoconjugates comprise the amino acid sequence of SEQ ID NO: 17. In some embodiments, the modified hIL-2 protein portion of the immunoconjugates comprise the amino acid sequence of SEQ ID NO: 18. In some embodiments, the modified hIL-2 protein portion of the immunoconjugates comprise the amino acid sequence of SEQ ID NO: 19. In some embodiments, the modified hIL-2 protein portion of the immunoconjugates comprise the amino acid sequence of SEQ ID NO: 20. In some embodiments, the modified hIL-2 protein portion of the immunoconjugates comprise the amino acid sequence of SEQ ID NO: 21. In some embodiments, the modified hIL-2 protein portion of the immunoconjugates comprise the amino acid sequence of SEQ ID NO: 22. In some embodiments, the modified hIL-2 protein portion of the immunoconjugates comprise the amino acid sequence of SEQ ID NO: 23. In some embodiments, the modified hIL-2 protein portion of the immunoconjugates comprise the amino acid sequence of SEQ ID NO: 24. In some embodiments, the modified hIL-2 protein portion of the immunoconjugates comprise the amino acid sequence of SEQ ID NO: 25. In some embodiments, the modified hIL-2 protein portion of the immunoconjugates comprise the amino acid sequence of SEQ ID NO: 26. In some embodiments, the modified hIL-2 protein portion of the immunoconjugates comprise the amino acid sequence of SEQ ID NO: 27. In some embodiments, the modified hIL-2 protein portion of the immunoconjugates comprise the amino acid sequence of SEQ ID NO: 28. In some embodiments, the modified hIL-2 protein portion of the immunoconjugates comprise the amino acid sequence of SEQ ID NO: 29. In some embodiments, the modified hIL-2 protein portion of the immunoconjugates comprise the amino acid sequence of SEQ ID NO: 30. In some embodiments, the modified hIL-2 protein portion of the immunoconjugates comprise the amino acid sequence of SEQ ID NO: 31. In some embodiments, the modified hIL-2 protein portion of the immunoconjugates comprise the amino acid sequence of SEQ ID NO: 32. In some embodiments, the modified hIL-2 protein portion of the immunoconjugates comprise the amino acid sequence of SEQ ID NO: 33. In some embodiments, the modified hIL-2 protein portion of the immunoconjugates comprise the amino acid sequence of SEQ ID NO: 34. In some embodiments, the modified hIL-2 protein portion of the immunoconjugates comprise the amino acid sequence of SEQ ID NO: 40. In some embodiments, the modified hIL-2 protein portion of the immunoconjugates comprise the amino acid sequence of SEQ ID NO: 41.

The modified hIL-2 protein of any one of amino acid sequences SEQ ID NOs: 10-34, 40, or 41 can further comprise a T3A substitution and/or a C125A substitution. In some embodiments, the modified hIL-2 protein portion of the immunoconjugates of any one of amino acid sequences SEQ ID NOs: 10-34, 40, or 41 further comprises a T3A substitution. In some embodiments, the modified hIL-2 protein portion of the immunoconjugates of any one of amino acid sequences SEQ ID NOs: 10-34, 40, or 41 further comprises a C125A substitution. In some embodiments, the modified hIL-2 protein portion of the immunoconjugates of any one of amino acid sequences SEQ ID NOs: 10-34, 40, or 41 further comprises a T3A substitution and a C125A substitution.

The modified hIL-2 protein portion of the immunoconjugates can comprise a D20A substitution relative to the non-modified hIL-2 amino acid sequence of SEQ ID NO: 42 and a R38E substitution relative to the non-modified hIL-2 amino acid sequence of SEQ ID NO: 42. In some embodiments, the modified hIL-2 protein portion of the immunoconjugates comprise the amino acid sequence of SEQ ID NO: 25. Thus, the stable pharmaceutical formulations can comprise:
about 18 mM to about 22 mM of acetate;
about 50 mM to about 250 mM of sucrose;
about 100 mM to about 500 mM of glycine;
about 0.05% to about 0.1% of PS80; and
about 5 mg/mL to about 10 mg/mL of an anti-hPD-1 antibody-modified hIL-2 immunoconjugate comprising a modified hIL-2 protein comprising the amino acid sequence of SEQ ID NO: 25.

The stable pharmaceutical formulations can comprise:
18 mM to 22 mM of acetate;
50 mM to 250 mM of sucrose;
100 mM to 500 mM of glycine;
0.05% to 0.1% of PS80; and
5 mg/mL to 10 mg/mL of an anti-hPD-1 antibody-modified hIL-2 immunoconjugate comprising a modified hIL-2 protein comprising the amino acid sequence of SEQ ID NO: 25.

The modified hIL-2 protein portion of the immunoconjugates can further comprise a substitution at amino acid position 3 relative to the non-modified hIL-2 amino acid sequence of SEQ ID NO: 42. A suitable substitution includes, for example, a T3A. In some embodiments, the modified hIL-2 protein portion of the immunoconjugates comprise a T3A substitution, a D20A substitution, and a R38E substitution. In some aspects, the modified hIL-2 protein portion of the immunoconjugates comprise the amino acid sequence of SEQ ID NO: 36.

Alternatively, the modified hIL-2 protein portion of the immunoconjugates can further comprise a deletion of amino acid position 3 relative to the non-modified hIL-2 amino acid sequence of SEQ ID NO: 42. In some embodiments, the modified hIL-2 protein portion of the immunoconjugates comprise a deletion of amino acids 1-3, a D20A substitution, and a R38E substitution. In some aspects, the modified hIL-2 protein portion of the immunoconjugates comprise the amino acid sequence of SEQ ID NO: 38.

The modified hIL-2 protein portion of the immunoconjugates can further comprise a deletion or substitution at amino acid position 125 relative to the non-modified hIL-2 amino acid sequence of SEQ ID NO: 42. The substitution at amino acid position 125 can be C125A. In some embodiments, the modified hIL-2 protein portion of the immunoconjugates comprise a D20A substitution, a R38E substitution, and a C125A substitution. In some embodiments, the modified hIL-2 protein portion of the immunoconjugates comprise the amino acid sequence of SEQ ID NO: 35. In some embodiments, the modified hIL-2 protein portion of the immunoconjugates comprise a T3A substitution, a D20A substitution, a R38E substitution, and a C125A substitution. In some embodiments, the modified hIL-2 protein portion of the immunoconjugates comprise the amino acid sequence of SEQ ID NO: 37. In some embodiments, the modified hIL-2 protein portion of the immunoconjugates comprise a deletion of amino acids 1-3, a D20A substitution, a R38E substitution, and a C125A substitution. In some embodiments, the modified hIL-2 protein portion of the immunoconjugates comprise the amino acid sequence of SEQ ID NO: 39.

The modified hIL-2 protein portion of the immunoconjugates can exhibit a reduction in potency of at least about 200-fold, at least about 500-fold, at least about 1,000-fold, at least about 2,000-fold, at least about 5,000-fold, at least about 6,500-fold, or at least about 10,000-fold on the high affinity IL-2 receptor (hIL-2Rαβγ) relative to a non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the high affinity IL-2 receptor (hIL-2Rαβγ) relative to a non-modified hIL-2. A greater reduction in hIL-2 potency on the high affinity hIL-2 receptor may be possible and acceptable for the modified hIL-2 proteins described herein, but such a reduction may not be quantifiable with the methods described herein due to limits of the cell proliferation assay conditions.

In addition, the modified hIL-2 protein portion of the immunoconjugates can exhibit a reduction in potency of at least about 200-fold, at least about 500-fold, at least about 1,000-fold, at least about 2,000-fold, at least about 5,000-fold, at least about 6,500-fold, or at least about 10,000-fold on the intermediate affinity IL-2 receptor (hIL-2Rβγ) relative to a non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the intermediate affinity IL-2 receptor (hIL-2Rβγ) relative to an non-modified hIL-2.

The modified hIL-2 protein portion of the immunoconjugates can exhibit a reduction in potency of up to about 10,000-fold on the high affinity IL-2 receptor (hIL-2Rαβγ) and a reduction in potency of up to about 10,000-fold on the intermediate affinity IL-2 receptor (hIL-2Rβγ) relative to a non-modified hIL-2, for example as quantified by a comparison in EC₅₀ values in an hIL-2-dependent cell proliferation assay described herein. The modified hIL-2 protein portion of the immunoconjugates can exhibit a reduction in potency of greater than about 10,000-fold on the high affinity IL-2 receptor (hIL-2Rαβγ) and a reduction in potency of greater than about 10,000-fold on the intermediate affinity IL-2 receptor (hIL-2Rβγ) relative to a non-modified hIL-2.

The anti-hPD-1 antibody-modified hIL-2 immunoconjugate can comprise an anti-hPD-1 antibody, or antigen-binding fragment thereof, that immunospecifically binds to hPD-1 and that comprises:
(i) a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76 (referred to herein as “H7-632”);
(ii) a heavy chain CDR1 comprising the amino acid sequence of SEQ ID NO: 45, a heavy chain CDR2 comprising the amino acid sequence of SEQ ID NO: 46, a heavy chain CDR3 comprising the amino acid sequence of SEQ ID NO: 47, a light chain CDR1 comprising the amino acid sequence of SEQ ID NO: 48, a light chain CDR2 comprising the amino acid sequence of SEQ ID NO: 49, and a light chain CDR3 comprising the amino acid sequence of SEQ ID NO: 50 (referred to herein as “2H7”);
(iii) a heavy chain CDR1 comprising the amino acid sequence of SEQ ID NO: 53, a heavy chain CDR2 comprising the amino acid sequence of SEQ ID NO: 54, a heavy chain CDR3 comprising the amino acid sequence of SEQ ID NO: 55, a light chain CDR1 comprising the amino acid sequence of SEQ ID NO: 56, a light chain CDR2 comprising the amino acid sequence of SEQ ID NO: 57, and a light chain CDR3 comprising the amino acid sequence of SEQ ID NO: 58 (referred to herein as “C51E6-5”); or
(iv) a heavy chain CDR1 comprising the amino acid sequence of SEQ ID NO: 61, a heavy chain CDR2 comprising the amino acid sequence of SEQ ID NO: 62, a heavy chain CDR3 comprising the amino acid sequence of SEQ ID NO: 63, a light chain CDR1 comprising the amino acid sequence of SEQ ID NO: 64, a light chain CDR2 comprising the amino acid sequence of SEQ ID NO: 65, and a light chain CDR3 comprising the amino acid sequence of SEQ ID NO: 66 (referred to herein as “A2”).

In some embodiments, the anti-hPD-1 antibody, or antigen-binding fragment thereof, portion of the immunoconjugates comprise a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76 (referred to herein as “H7-632”). The stable pharmaceutical formulations can comprise, for example:
about 18 mM to about 22 mM of acetate;
about 50 mM to about 250 mM of sucrose;
about 100 mM to about 500 mM of glycine;
about 0.05% to about 0.1% of PS80; and
about 5 mg/mL to about 10 mg/mL of an anti-hPD-1 antibody-modified hIL-2 immunoconjugate comprising:
a modified hIL-2 protein comprising the amino acid sequence of SEQ ID NO: 25; and
an anti-hPD-1 antibody, or antigen-binding fragment thereof, that immunospecifically binds to hPD-1, wherein the antibody or antigen-binding fragment thereof, comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.

The stable pharmaceutical formulations can comprise, for example:
18 mM to 22 mM of acetate;
50 mM to 250 mM of sucrose;
100 mM to 500 mM of glycine;
0.05% to 0.1% of PS80; and
5 mg/mL to 10 mg/mL of an anti-hPD-1 antibody-modified hIL-2 immunoconjugate comprising:
a modified hIL-2 protein comprising the amino acid sequence of SEQ ID NO: 25; and
an anti-hPD-1 antibody, or antigen-binding fragment thereof, that immunospecifically binds to hPD-1, wherein the antibody or antigen-binding fragment thereof, comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.

In some embodiments, the anti-hPD-1 antibody, or antigen-binding fragment thereof, portion of the immunoconjugates comprise a heavy chain CDR1 comprising the amino acid sequence of SEQ ID NO: 45, a heavy chain CDR2 comprising the amino acid sequence of SEQ ID NO: 46, a heavy chain CDR3 comprising the amino acid sequence of SEQ ID NO: 47, a light chain CDR1 comprising the amino acid sequence of SEQ ID NO: 48, a light chain CDR2 comprising the amino acid sequence of SEQ ID NO: 49, and a light chain CDR3 comprising the amino acid sequence of SEQ ID NO: 50 (referred to herein as “2H7”).

In some embodiments, the anti-hPD-1 antibody, or antigen-binding fragment thereof, portion of the immunoconjugates comprise a heavy chain CDR1 comprising the amino acid sequence of SEQ ID NO: 53, a heavy chain CDR2 comprising the amino acid sequence of SEQ ID NO: 54, a heavy chain CDR3 comprising the amino acid sequence of SEQ ID NO: 55, a light chain CDR 1 comprising the amino acid sequence of SEQ ID NO: 56, a light chain CDR2 comprising the amino acid sequence of SEQ ID NO: 57, and a light chain CDR3 comprising the amino acid sequence of SEQ ID NO: 58 (referred to herein as “C51E6-5”).

In some embodiments, the anti-hPD-1 antibody, or antigen-binding fragment thereof, portion of the immunoconjugates comprise a heavy chain CDR1 comprising the amino acid sequence of SEQ ID NO: 61, a heavy chain CDR2 comprising the amino acid sequence of SEQ ID NO: 62, a heavy chain CDR3 comprising the amino acid sequence of SEQ ID NO: 63, a light chain CDR1 comprising the amino acid sequence of SEQ ID NO: 64, a light chain CDR2 comprising the amino acid sequence of SEQ ID NO: 65, and a light chain CDR3 comprising the amino acid sequence of SEQ ID NO: 66 (referred to herein as “A2”).

The anti-hPD-1 antibody, or antigen-binding fragment thereof, portion of the immunoconjugates can comprise a heavy chain variable region (VH) comprising the amino acid sequence of SEQ ID NO: 69 and a light chain variable region (VL) comprising the amino acid sequence of SEQ ID NO: 70 (referred to herein as “H7-632”).

The anti-hPD-1 antibody, or antigen-binding fragment thereof, portion of the immunoconjugates can comprise a VH comprising the amino acid sequence of SEQ ID NO: 43 and a VL comprising the amino acid sequence of SEQ ID NO: 44 (referred to herein as “2H7”).

The anti-hPD-1 antibody, or antigen-binding fragment thereof, portion of the immunoconjugates can comprise a VH comprising the amino acid sequence of SEQ ID NO: 51 and a VL comprising the amino acid sequence of SEQ ID NO: 52 (referred to herein as “C51E6-5”).

The anti-hPD-1 antibody, or antigen-binding fragment thereof, portion of the immunoconjugates can comprise a VH comprising the amino acid sequence of SEQ ID NO: 59 and a VL comprising the amino acid sequence of SEQ ID NO: 60 (referred to herein as “A2”).

The anti-hPD-1 antibody, or antigen-binding fragment thereof, portion of the immunoconjugates can comprise an IgG1 heavy chain constant region.

The anti-hPD-1 antibody, or antigen-binding fragment thereof, portion of the immunoconjugates can have substitutions or deletions within the constant region to minimize Fc-mediated immune effector function, such as FcγRIIIA-mediated antibody-dependent cell-mediated cytotoxicity (ADCC), FcγRI- and FcγRIIa-dependent antibody-dependent cellular phagocytosis (ADCP), and C1q binding-mediated complement-dependent cytotoxicity (CDC). In some embodiments, the anti-hPD-1 antibody, or antigen-binding fragment thereof, portion of the immunoconjugates comprises a L235A substitution, wherein the amino acid numbering is according to EU numbering. In some embodiments, the anti-hPD-1 antibody, or antigen-binding fragment thereof, portion of the immunoconjugates comprises a G237A substitution, wherein the amino acid numbering is according to EU numbering. In some embodiments, the anti-hPD-1 antibody, or antigen-binding fragment thereof, portion of the immunoconjugates comprises an L235A substitution and a G237A substitution, wherein the amino acid numbering is according to EU numbering.

The anti-hPD-1 antibody, or antigen-binding fragment thereof, portion of the immunoconjugates can comprise a heavy chain (HC) comprising the amino acid sequence of SEQ ID NO: 67 and a light chain (LC) comprising the amino acid sequence of SEQ ID NO: 68 (referred to herein as “H7-632-hIgG1-LAGA”).

The anti-hPD-1 antibody, or antigen-binding fragment thereof, portion of the immunoconjugate can comprise a HC comprising the amino acid sequence of SEQ ID NO: 77 and a LC comprising the amino acid sequence of SEQ ID NO: 78 (referred to herein as “2H7-hIgG4”).

The anti-hPD-1 antibody, or antigen-binding fragment thereof, portion of the immunoconjugate can comprise a HC comprising the amino acid sequence of SEQ ID NO: 79 and a LC comprising the amino acid sequence of SEQ ID NO: 80 (referred to herein as “C51E6-5-hIgG4”).

The anti-hPD-1 antibody, or antigen-binding fragment thereof, portion of the immunoconjugate can comprise a HC comprising the amino acid sequence of SEQ ID NO: 81 and a LC comprising the amino acid sequence of SEQ ID NO: 82 (referred to herein as “A2-hIgG4”).

Disclosed herein are stable pharmaceutical formulations comprising:
about 18 mM to about 22 mM of acetate;
about 50 mM to about 250 mM of sucrose;
about 100 mM to about 500 mM of glycine;
about 0.05% to about 0.1% of PS80; and about 5 mg/mL to about 10 mg/mL of an anti-hPD-1 antibody-modified hIL-2 immunoconjugate comprising:
   a modified hIL-2 protein comprising the amino acid sequence of SEQ ID NO: 25; and
   an anti-hPD-1 antibody, or antigen-binding fragment thereof, that immunospecifically binds to hPD-1, wherein the antibody or antigen-binding fragment thereof, comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.

The stable pharmaceutical formulations can comprise 18 mM to 22 mM of acetate, 50 mM to 250 mM of sucrose, 100 mM to 500 mM of glycine, 0.05% to 0.1% of PS80, and 5 mg/mL to 10 mg/mL of an anti-hPD-1 antibody-modified hIL-2 immunoconjugate.

The stable pharmaceutical formulations can comprise about 20 mM of acetate, about 200 mM of sucrose, about 400 mM of glycine, and about 0.05% of PS80. The stable pharmaceutical formulations can comprise 20 mM of acetate, 200 mM of sucrose, 400 mM of glycine, and 0.05% of PS80.

The stable pharmaceutical formulations can comprise about 10 mg/mL of the anti-hPD-1 antibody-modified hIL-2 immunoconjugate. The stable pharmaceutical formulations can comprise 10 mg/mL of the anti-hPD-1 antibody-modified hIL-2 immunoconjugate.

The stable pharmaceutical formulations can have a pH of about 4.5 to about 5.5. The stable pharmaceutical formulations can have a pH of 4.5 to 5.5. In some embodiments, the stable pharmaceutical formulations have a pH of about 5.0. In some embodiments, the stable pharmaceutical formulations have a pH of 5.0.

The modified hIL-2 protein can further comprise a deletion or substitution at amino acid position 3. In some embodiments, the substitution at amino acid position 3 of the modified hIL-2 protein is T3A. In some embodiments, the modified hIL-2 protein comprises the amino acid sequence of SEQ ID NO: 36.

The modified hIL-2 protein can further comprise a deletion or substitution at amino acid position 125. In some embodiments, the substitution at amino acid position 125 is C125A. In some embodiments, the modified hIL-2 protein comprises the amino acid sequence of SEQ ID NO: 37.

The anti-hPD-1 antibody, or antigen-binding fragment thereof, can comprise a VH comprising the amino acid sequence of SEQ ID NO: 69 and a VL comprising the amino acid sequence of SEQ ID NO: 70.

The anti-hPD-1 antibody, or antigen-binding fragment thereof, can comprise an IgG1 heavy chain constant region. In some embodiments, the anti-hPD-1 antibody, or antigen-binding fragment thereof, comprises an L235A substitution and a G237A substitution, according to EU numbering. In some embodiments, the anti-hPD-1 antibody, or antigen-binding fragment thereof, comprises a HC comprising the amino acid sequence of SEQ ID NO: 67 and a LC comprising the amino acid sequence of SEQ ID NO: 68. Thus, disclosed herein are stable pharmaceutical formulations comprising:
about 18 mM to about 22 mM of acetate;
about 50 mM to about 250 mM of sucrose;
about 100 mM to about 500 mM of glycine;
about 0.05% to about 0.1% of PS80; and
about 5 mg/mL to about 10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a HC comprising the amino acid sequence of SEQ ID NO: 67 and a LC comprising the amino acid sequence of SEQ ID NO: 68.

The stable pharmaceutical formulations can comprise 18 mM to 22 mM of acetate, 50 mM to 250 mM of sucrose, 100 mM to 500 mM of glycine, 0.05% to 0.1% of PS80, and 5 mg/mL to 10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a HC comprising the amino acid sequence of SEQ ID NO: 67 and a LC comprising the amino acid sequence of SEQ ID NO: 68.

The stable pharmaceutical formulation can comprise about 10 mg/mL of the hPD-1 antibody-modified hIL-2 immunoconjugate, about 20 mM of acetate, about 200 mM of sucrose, about 400 mM of glycine, and about 0.05% of PS80, and a pH of about 5.0. Thus, the stable pharmaceutical formulations can comprise:
about 20 mM of acetate;
about 200 mM of sucrose;
about 400 mM of glycine;
about 0.05% of PS80; and
about 10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a heavy chain comprising the amino acid sequence of SEQ ID NO: 67 and a light chain comprising the amino acid sequence of SEQ ID NO: 68,
wherein the liquid pharmaceutical formulation has a pH of about 5.0.

The stable pharmaceutical formulations can comprise 20 mM of acetate, 200 mM of sucrose, 400 mM of glycine, 0.05% of PS80, and 10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a heavy chain comprising the amino acid sequence of SEQ ID NO: 67 and a light chain comprising the amino acid sequence of SEQ ID NO: 68, wherein the liquid pharmaceutical formulation has a pH of about 5.0.

The immunoconjugate can comprise a modified hIL-2 protein comprising a T3A substitution, a D20A substitution, a R38E substitution, and a C125A substitution fused to the C-terminus of the anti-hPD-1 antibody heavy chain comprising a human IgG1 framework with a L235A substitution and a G237A substitution. In some embodiments, the stable pharmaceutical formulations can comprise an anti-hPD-1 antibody-modified hIL-2 immunoconjugate that comprises a light chain comprising the amino acid sequence of SEQ ID NO: 68 and a heavy chain-modified hIL-2 protein fusion comprising the amino acid sequence of SEQ ID NO: 83. Thus, disclosed herein are stable pharmaceutical formulations comprising:
about 18 mM to about 22 mM of acetate;
about 50 mM to about 250 mM of sucrose;
about 100 mM to about 500 mM of glycine;
about 0.05% to about 0.1% of PS80; and about 5 mg/mL to about 10 mg/mL of an anti-hPD-1 antibody-modified hIL-2 immunoconjugate comprising a light chain comprising the amino acid sequence of SEQ ID NO: 68 and a heavy chain-modified hIL-2 protein fusion comprising the amino acid sequence of SEQ ID NO: 83,
wherein the liquid pharmaceutical formulation has a pH of about 5.0.

The pharmaceutical formulations can comprise 18 mM to 22 mM of acetate, 50 mM to 250 mM of sucrose, 100 mM to 500 mM of glycine, 0.05% to 0.1% of PS80, and 5 mg/mL to 10 mg/mL of an anti-hPD-1 antibody-modified hIL-2 immunoconjugate comprising a light chain comprising the amino acid sequence of SEQ ID NO: 68 and a heavy chain-modified hIL-2 protein fusion comprising the amino acid sequence of SEQ ID NO: 83, wherein the liquid pharmaceutical formulation has a pH of 5.0.

In some embodiments, the anti-hPD-1 antibody-modified hIL-2 immunoconjugate is H7-767 (also referred to herein as H7-632-IgG1-LAGA-IL-2AAEA).

Disclosed herein are stable lyophilized pharmaceutical formulations prepared by lyophilizing any of the disclosed stable pharmaceutical formulations. In some embodiments, the stable lyophilized pharmaceutical formulations can comprise:
acetate;
sucrose;
glycine;
PS80; and
an anti-hPD-1 antibody-modified hIL-2 immunoconjugate comprising:
   a modified hIL-2 protein comprising the amino acid sequence of SEQ ID NO: 25; and
   an anti-hPD-1 antibody, or antigen-binding fragment thereof, that immunospecifically binds to hPD-1, wherein the antibody or antigen-binding fragment thereof comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.

Once reconstituted, the stable lyophilized pharmaceutical formulations can comprise about 10 mg/mL of the hPD-1 antibody-modified hIL-2 immunoconjugate, about 20 mM of acetate, about 200 mM of sucrose, about 400 mM of glycine, and about 0.05% of PS80, with a pH of about 5.0. In some embodiments, once reconstituted, the stable lyophilized pharmaceutical formulations can comprise 10 mg/mL of the hPD-1 antibody-modified hIL-2 immunoconjugate, 20 mM of acetate, 200 mM of sucrose, 400 mM of glycine, and 0.05% of PS80, with a pH of 5.0.

The anti-hPD-1 antibody-modified hIL-2 immunoconjugate can have one or more of the following properties:
Binds to PD-1 but does not inhibit PD-L1 binding to PD-1;
Binds to PD-1 in the presence of standard-of-care anti-PD-1 antibodies used in the clinic (e.g., pembrolizumab (KEYTRUDA®) or nivolumab (OPDIVO®));
Is highly selective for PD-1 and does not immunospecifically bind other related B7 family members;
Binds PD-1 on activated human T cells (EC₅₀˜0.1-0.2 nM in a flow binding assay);
The modified hIL-2 protein portion of the immunoconjugate demonstrates a measurable reduction in potency of at least about 200-fold, at least about 500-fold, at least about 1,000-fold, at least about 2,000-fold, at least about 5,000-fold, at least about 6,500-fold, or at least about 10,000-fold on the high affinity IL-2 receptor (hIL-2Rαβγ) relative to a non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the high affinity IL-2 receptor (hIL-2Rαβγ) relative to a non-modified hIL-2. A greater reduction in hIL-2 potency on the high affinity hIL-2 receptor may be possible and acceptable for the modified hIL-2 proteins described herein, but such a reduction may not be quantifiable with the methods described herein due to limits of the cell proliferation assay conditions;
The modified hIL-2 protein portion of the immunoconjugate demonstrates a measurable reduction in potency of at least about 200-fold, at least about 500-fold, at least about 1,000-fold, at least about 2,000-fold, at least about 5,000-fold, at least about 6,500-fold, or at least about 10,000-fold on the intermediate affinity IL-2 receptor (hIL-2Rβγ) relative to an non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the intermediate affinity IL-2 receptor (hIL-2Rβγ) relative to a non-modified hIL-2;
Rescues and expands PD-1-expressing human memory T cell subsets in a GvHD animal model; and
Has minimal or no impact on body weight, blood chemistry, or hematology parameters after single dose at 1 mg/kg and 10 mg/kg in cynomolgus monkeys.

The disclosed immunoconjugates can selectively deliver IL-2 signaling to PD-1-expressing T cells. The anti-hPD-1 antibody, or antigen-binding fragment thereof, portion of the immunoconjugate can be utilized solely to deliver the modified hIL-2 to PD-1-expressing cells and does not block PD-1 receptor function, as do classical anti-PD-1 inhibitor antibodies such as nivolumab (OPDIVO®) and pembrolizumab (KEYTRUDA®). The primary mechanism-of-action of the herein disclosed immunoconjugates is via the T cell selective activity of IL-2. The human PD-1 receptor is primarily expressed on a minor subset of T cells with potent tumor reactivity. Without being bound by theory, it is believed that targeting the modified hIL-2 protein portion of the immunoconjugate to this population of T cells can dramatically amplify anti-tumor immunity while reducing or minimizing off-target systemic IL-2-mediated toxicities mediated by cell populations that lack PD-1 expression.

The modified hIL-2 protein can be fused to the anti-hPD-1 antibody, or antigen-binding fragment thereof, portion of the immunoconjugate at the N-terminus of an antibody light chain, the C-terminus of an antibody light chain, the N-terminus of an antibody heavy chain, the C-terminus of an antibody heavy chain, the N-terminus of the antigen-binding fragment, or the C-terminus of the antigen-binding fragment. In some embodiments, the modified hIL-2 protein portion of the immunoconjugates is directly fused by a peptide bond to the anti-hPD-1 antibody, or antigen-binding fragment thereof, portion of the immunoconjugate. The modified hIL-2 protein portion of the immunoconjugates can be, for example, directly fused by a peptide bond to the C-terminal amino acid residue of the anti-hPD-1 antibody, or antigen-binding fragment thereof, portion of the immunoconjugate. In some embodiments, the modified hIL-2 protein is fused to the anti-hPD-1 antibody, or antigen-binding fragment thereof, portion of the immunoconjugate through a linker.

Fusion of the modified hIL-2 proteins to the anti-hPD-1 antibody, or antigen-binding fragment thereof, portion of the immunoconjugate can rescue the modified hIL-2 proteins' ability to activate the intermediate affinity IL-2 receptor. In some embodiments, the immunoconjugate is able to activate the intermediate affinity IL-2 receptor to a degree that is comparable to wild type hIL-2 activation of the intermediate affinity IL-2 receptor.

Disclosed herein are methods of treating a cancer in a subject, the methods comprising administering a therapeutically effective amount of any of the disclosed stable pharmaceutical formulations to the subject to thereby treat the cancer. In some embodiments, the stable pharmaceutical formulations can comprise about 10 mg/mL of the hPD-1 antibody-modified hIL-2 immunoconjugate, about 20 mM of acetate, about 200 mM of sucrose, about 400 mM of glycine, and about 0.05% of PS80, and has a pH of about 5.0. In some embodiments, the stable pharmaceutical formulations can comprise 10 mg/mL of the hPD-1 antibody-modified hIL-2 immunoconjugate, 20 mM of acetate, 200 mM of sucrose, 400 mM of glycine, and 0.05% of PS80, and has a pH of 5.0.

The stable pharmaceutical formulations may be administered to subjects who are beginning to show signs of relapse/recurrence after a period of antagonistic PD-1 therapy. Thus, in some embodiments, the methods are performed on subjects who are beginning to show signs of relapse/recurrence after or during antagonistic PD-1 therapy.

Exemplary cancers that can be treated using the disclosed methods include bladder cancer, brain cancer, head and neck cancer, pancreatic cancer, lung cancer, non-small cell lung carcinoma, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, melanoma, squamous cell carcinoma, bone cancer, and kidney cancer. In some embodiments, the disclosed methods can be used to treat a melanoma, a Merkel cell carcinoma, a non-small cell lung carcinoma, a renal cell carcinoma, a triple negative breast cancer, a squamous cell carcinoma of the head/neck, a hepatocellular carcinoma, or microsatellite instability-high tumors or tumors with deficient DNA mismatch repair.

Each and every aspect of the disclosed and claimed methods of treatment are equally applicable to uses of the stable pharmaceutical formulations, including, but not limited to, Swiss-style uses, first medical uses, second/further medical uses, and uses in medicine. Thus, for example, disclosed herein are uses of stable pharmaceutical formulations comprising:
about 18 mM to about 22 mM of acetate;
about 50 mM to about 250 mM of sucrose;
about 100 mM to about 500 mM of glycine;
about 0.05% to about 0.1% of PS80; and
about 5 mg/mL to about 10 mg/mL of an anti-hPD-1 antibody-modified hIL-2 immunoconjugate comprising:
   a modified hIL-2 protein comprising the amino acid sequence of SEQ ID NO: 25; and
   an anti-hPD-1 antibody, or antigen-binding fragment thereof, that immunospecifically binds to hPD-1, wherein the antibody or antigen-binding fragment thereof, comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76, for the treatment of cancer.

Also disclosed herein are uses of stable pharmaceutical formulations comprising:
18 mM to 22 mM of acetate;
50 mM to 250 mM of sucrose;
100 mM to 500 mM of glycine;
0.05% to 0.1% of PS80; and
5 mg/mL to 10 mg/mL of an anti-hPD-1 antibody-modified hIL-2 immunoconjugate comprising:
   a modified hIL-2 protein comprising the amino acid sequence of SEQ ID NO: 25; and
   an anti-hPD-1 antibody, or antigen-binding fragment thereof, that immunospecifically binds to hPD-1, wherein the antibody or antigen-binding fragment thereof, comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76,
for the treatment of cancer.

### Embodiments

Provided below is a list of embodiments, which are intended to complement, rather than displace or supersede, the previous descriptions.
1a. A stable pharmaceutical formulation comprising:
   about 18 mM to about 22 mM of acetate;
   about 50 mM to about 250 mM of sucrose;
   about 100 mM to about 500 mM of glycine;
   about 0.05% to about 0.1% of PS80; and
   about 5 mg/mL to about 10 mg/mL of an anti-human PD-1 (hPD-1) antibody-modified human interleukin-2 (hIL-2) immunoconjugate comprising:
a modified hIL-2 protein comprising the amino acid sequence of SEQ ID NO: 25; and
   an anti-hPD-1 antibody, or antigen-binding fragment thereof, that immunospecifically binds to hPD-1, wherein the antibody or antigen-binding fragment thereof, comprises a heavy chain complementarity determining region 1 (CDR1) comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.
2a. The stable pharmaceutical formulation of embodiment 1a, comprising:
   about 20 mM of acetate;
   about 200 mM of sucrose;
   about 400 mM of glycine; and
   about 0.05% of PS80.
3a. The stable pharmaceutical formulation of embodiment 1a or 2a, comprising about 10 mg/mL of the anti-hPD-1 antibody-modified hIL-2 immunoconjugate.
4a. The stable pharmaceutical formulation of any one of the previous embodiments, wherein the pH is about 4.5 to about 5.5.
5a. The stable pharmaceutical formulation of embodiment 4a, wherein the pH is about 5.0.
6a. The stable pharmaceutical formulation of any one of the previous embodiments, wherein the modified hIL-2 protein further comprises a deletion or substitution at amino acid position 3.
7a. The stable pharmaceutical formulation of embodiment 6a, wherein the substitution at amino acid position 3 of the modified hIL-2 protein is T3A.
8a. The stable pharmaceutical formulation of embodiment 7a, wherein the modified hIL-2 protein comprises the amino acid sequence of SEQ ID NO: 36.
9a. The stable pharmaceutical formulation of any one of the previous embodiments, wherein the modified hIL-2 protein further comprises a deletion or substitution at amino acid position 125.
10a. The stable pharmaceutical formulation of embodiment 9a, wherein the substitution at amino acid position 125 is C125A.
11a. The stable pharmaceutical formulation of embodiment 10a, wherein the modified hIL-2 protein comprises the amino acid sequence of SEQ ID NO: 37.
12a. The stable pharmaceutical formulation of any one of the previous embodiments, wherein the anti-hPD-1 antibody, or antigen-binding fragment thereof, comprises a heavy chain variable region (VH) comprising the amino acid sequence of SEQ ID NO: 69 and a light chain variable region (VL) comprising the amino acid sequence of SEQ ID NO: 70.
13a. The stable pharmaceutical formulation of any one of the previous embodiments, wherein the anti-hPD-1 antibody, or antigen-binding fragment thereof, comprises an IgG1 heavy chain constant region.
14a. The stable pharmaceutical formulation of embodiment 13a, wherein the anti-hPD-1 antibody, or antigen-binding fragment thereof, comprises an L235A substitution and a G237A substitution, according to EU numbering.
15a. The stable pharmaceutical formulation of any one of the previous embodiments, wherein the anti-hPD-1 antibody, or antigen-binding fragment thereof, comprises a heavy chain (HC) comprising the amino acid sequence of SEQ ID NO: 67 and a light chain (LC) comprising the amino acid sequence of SEQ ID NO: 68.
16a. The stable pharmaceutical formulation of any one of the previous embodiments, comprising:
   about 18 mM to about 22 mM of acetate;
   about 50 mM to about 250 mM of sucrose;
   about 100 mM to about 500 mM of glycine;
   about 0.05% to about 0.1% of PS80; and
   about 5 mg/mL to about 10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a HC comprising the amino acid sequence of SEQ ID NO: 67 and a LC comprising the amino acid sequence of SEQ ID NO: 68.
17a. The stable pharmaceutical formulation of any one of the previous embodiments, wherein the anti-hPD-1 antibody-modified hIL-2 immunoconjugate comprises:
   a light chain comprising the amino acid sequence of SEQ ID NO: 68; and
   a heavy chain-modified hIL-2 protein fusion comprising the amino acid sequence of SEQ ID NO: 83.
18a. The stable pharmaceutical formulation of any one of the previous embodiments, comprising about 10 mg/mL of the hPD-1 antibody-modified hIL-2 immunoconjugate, about 20 mM of acetate, about 200 mM of sucrose, about 400 mM of glycine, and about 0.05% of PS80, and a pH of about 5.0.
19a. The stable pharmaceutical formulation of any one of the previous embodiments, comprising:
   about 20 mM of acetate;
   about 200 mM of sucrose;
   about 400 mM of glycine;
   about 0.05% of PS80; and
   about 10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a heavy chain comprising the amino acid sequence of SEQ ID NO: 67 and a light chain comprising the amino acid sequence of SEQ ID NO: 68,
   wherein the liquid pharmaceutical formulation has a pH of about 5.0.
20a. A stable lyophilized pharmaceutical formulation prepared by lyophilizing the stable pharmaceutical formulation of any one of embodiments 1a-19a.
21a. The stable lyophilized pharmaceutical formulation of embodiment 20a, comprising:
   acetate;
   sucrose;
   glycine;
   PS80; and
   an anti-hPD-1 antibody-modified hIL-2 immunoconjugate comprising:
      a modified hIL-2 protein comprising the amino acid sequence of SEQ ID NO: 25; and
      an anti-hPD-1 antibody, or antigen-binding fragment thereof, that immunospecifically binds to hPD-1, wherein the antibody or antigen-binding fragment thereof comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.
22a. The stable lyophilized pharmaceutical formulation of embodiment 21a, wherein, once reconstituted, comprises about 10 mg/mL of the hPD-1 antibody-modified hIL-2 immunoconjugate, about 20 mM of acetate, about 200 mM of sucrose, about 400 mM of glycine, and about 0.05% of PS80, and has a pH of about 5.0.
23a. A method of treating a cancer in a subject, the method comprising:
   administering a therapeutically effective amount of the stable pharmaceutical formulation of any one of embodiments 1a-19a to the subject to thereby treat the cancer.
24a. The method of embodiment 23a, wherein the stable pharmaceutical formulation comprises about 10 mg/mL of the hPD-1 antibody-modified hIL-2 immunoconjugate, about 20 mM of acetate, about 200 mM of sucrose, about 400 mM of glycine, and about 0.05% of PS80, and has a pH of about 5.0.
25a. The method of embodiment 23a or 24a, wherein the cancer is a melanoma, a Merkel cell carcinoma, a non-small cell lung carcinoma, a renal cell carcinoma, a triple negative breast cancer, a squamous cell carcinoma of the head/neck, a hepatocellular carcinoma, or microsatellite instability-high tumors or tumors with deficient DNA mismatch repair.

Provided below is a list of additional embodiments, which are intended to complement, rather than displace or supersede, the previous descriptions.
1b. A stable pharmaceutical formulation comprising:
   18 mM to 22 mM of acetate;
   50 mM to 250 mM of sucrose;
   100 mM to 500 mM of glycine;
   0.05% to 0.1% of PS80; and
   5 mg/mL to 10 mg/mL of an anti-human PD-1 (hPD-1) antibody-modified human interleukin-2 (hIL-2) immunoconjugate comprising:
      a modified hIL-2 protein comprising the amino acid sequence of SEQ ID NO: 25; and
      an anti-hPD-1 antibody, or antigen-binding fragment thereof, that immunospecifically binds to hPD-1, wherein the antibody or antigen-binding fragment thereof, comprises a heavy chain complementarity determining region 1 (CDR1) comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.
2b. The stable pharmaceutical formulation of embodiment 1b, comprising:
   20 mM of acetate;
   200 mM of sucrose;
   400 mM of glycine; and
   0.05% of PS80.
3b. The stable pharmaceutical formulation of embodiment 1b or 2b, comprising 10 mg/mL of the anti-hPD-1 antibody-modified hIL-2 immunoconjugate.
4b. The stable pharmaceutical formulation of any one of the previous embodiments, wherein the pH is 4.5 to 5.5.
5b. The stable pharmaceutical formulation of embodiment 4b, wherein the pH is 5.0.
6b. The stable pharmaceutical formulation of any one of the previous embodiments, wherein the modified hIL-2 protein further comprises a deletion or substitution at amino acid position 3.
7b. The stable pharmaceutical formulation of embodiment 6b, wherein the substitution at amino acid position 3 of the modified hIL-2 protein is T3A.
8b. The stable pharmaceutical formulation of embodiment 7b, wherein the modified hIL-2 protein comprises the amino acid sequence of SEQ ID NO: 36.
9b. The stable pharmaceutical formulation of any one of the previous embodiments, wherein the modified hIL-2 protein further comprises a deletion or substitution at amino acid position 125.
10b. The stable pharmaceutical formulation of embodiment 9b, wherein the substitution at amino acid position 125 is C125A.
11b. The stable pharmaceutical formulation of embodiment 10b, wherein the modified hIL-2 protein comprises the amino acid sequence of SEQ ID NO: 37.
12b. The stable pharmaceutical formulation of any one of the previous embodiments, wherein the anti-hPD-1 antibody, or antigen-binding fragment thereof, comprises a heavy chain variable region (VH) comprising the amino acid sequence of SEQ ID NO: 69 and a light chain variable region (VL) comprising the amino acid sequence of SEQ ID NO: 70.
13b. The stable pharmaceutical formulation of any one of the previous embodiments, wherein the anti-hPD-1 antibody, or antigen-binding fragment thereof, comprises an IgG1 heavy chain constant region.
14b. The stable pharmaceutical formulation of embodiment 13b, wherein the anti-hPD-1 antibody, or antigen-binding fragment thereof, comprises an L235A substitution and a G237A substitution, according to EU numbering.
15b. The stable pharmaceutical formulation of any one of the previous embodiments, wherein the anti-hPD-1 antibody, or antigen-binding fragment thereof, comprises a heavy chain (HC) comprising the amino acid sequence of SEQ ID NO: 67 and a light chain (LC) comprising the amino acid sequence of SEQ ID NO: 68.
16b. The stable pharmaceutical formulation of any one of the previous embodiments, comprising:
   18 mM to 22 mM of acetate;
   50 mM to 250 mM of sucrose;
   100 mM to 500 mM of glycine;
   0.05% to 0.1% of PS80; and
   5 mg/mL to 10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a HC comprising the amino acid sequence of SEQ ID NO: 67 and a LC comprising the amino acid sequence of SEQ ID NO: 68.
17b. The stable pharmaceutical formulation of any one of the previous embodiments, wherein the anti-hPD-1 antibody-modified hIL-2 immunoconjugate comprises:
   a light chain comprising the amino acid sequence of SEQ ID NO: 68; and
   a heavy chain-modified hIL-2 protein fusion comprising the amino acid sequence of SEQ ID NO: 83.
18b. The stable pharmaceutical formulation of any one of the previous embodiments, comprising 10 mg/mL of the hPD-1 antibody-modified hIL-2 immunoconjugate, 20 mM of acetate, 200 mM of sucrose, 400 mM of glycine, and 0.05% of PS80, and a pH of 5.0.
19b. The stable pharmaceutical formulation of any one of the previous embodiments, comprising:
   20 mM of acetate;
   200 mM of sucrose;
   400 mM of glycine;
   0.05% of PS80; and
   10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a heavy chain comprising the amino acid sequence of SEQ ID NO: 67 and a light chain comprising the amino acid sequence of SEQ ID NO: 68,
   wherein the liquid pharmaceutical formulation has a pH of 5.0.
20b. A stable lyophilized pharmaceutical formulation prepared by lyophilizing the stable pharmaceutical formulation of any one of embodiments 1b-19b.
21b. The stable lyophilized pharmaceutical formulation of embodiment 20b, comprising:
   acetate;
   sucrose;
   glycine;
   PS80; and
   an anti-hPD-1 antibody-modified hIL-2 immunoconjugate comprising:
      a modified hIL-2 protein comprising the amino acid sequence of SEQ ID NO: 25; and
      an anti-hPD-1 antibody, or antigen-binding fragment thereof, that immunospecifically binds to hPD-1, wherein the antibody or antigen-binding fragment thereof comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.
22b. The stable lyophilized pharmaceutical formulation of embodiment 21b, wherein, once reconstituted, comprises 10 mg/mL of the hPD-1 antibody-modified hIL-2 immunoconjugate, 20 mM of acetate, 200 mM of sucrose, 400 mM of glycine, and 0.05% of PS80, and has a pH of 5.0
23b. A method of treating a cancer in a subject, the method comprising:
   administering a therapeutically effective amount of the stable pharmaceutical formulation of any one of embodiments 1b-19b to the subject to thereby treat the cancer.
24b. The method of embodiment 23b, wherein the stable pharmaceutical formulation comprises 10 mg/mL of the hPD-1 antibody-modified hIL-2 immunoconjugate, 20 mM of acetate, 200 mM of sucrose, 400 mM of glycine, and 0.05% of PS80, and has a pH of 5.0.
25b. The method of embodiment 23b or 24b, wherein the cancer is a melanoma, a Merkel cell carcinoma, a non-small cell lung carcinoma, a renal cell carcinoma, a triple negative breast cancer, a squamous cell carcinoma of the head/neck, a hepatocellular carcinoma, or microsatellite instability-high tumors or tumors with deficient DNA mismatch repair.

### Examples

The following examples are provided to further describe some of the embodiments disclosed herein. The examples are intended to illustrate, not to limit, the disclosed embodiments.

### Liquid Formulation Development

### General Methods

### DLS (Dynamic Light Scattering)

A sample of each test formulation was centrifuged at 10,000 RPM for 10 minutes to remove any large particulates. Without disturbing the centrifuged solution, 30 μL of each sample was transferred to a clear microplate (Corning, Inc; 3540) in triplicates and covered using an adhesive sealing film. To remove any bubbles, the microplate was centrifuged at 3000 RPM for 5 minutes. The sealing film was removed, and the plate was loaded to the Wyatt DynaPro Plate Reader™ II using the software version of 7.9.1 and control firmware version of 2.0.6.15.

Determination of hydrodynamic radius (Rh) polydispersity (PD) %) of H7-767—To obtain the measurements on Rh (nm) and PD (%) of H7-767 (immunoconjugate having the H7-767 HC of SEQ ID NO: 83 and the H7-632 LC of SEQ ID NO: 68), the experiment was set up as “isothermal” at 25° C. for 10 acquisitions at 10 seconds of acquisition time with 60 seconds of auto-attenuation. One measurement per well with a scan was performed without a wait time between measurements within a scan. Laser wavelength was set as 825 nm at 150° detector angle. The correlation function low and high cutoffs were set at 1.5 us and 60000 μs, respectively. The low and high peak radius cutoffs were set at 0.5 nm and 10000 nm. The measurement time limit factor was 5.0 and minimum peak intensity was 1%.

Determination of diffusion interaction parameter (kD) of H7-767—The H7-767 formulations were prepared at 7 different protein concentrations (20 mg/mL, 10 mg/mL, 8 mg/mL, 6 mg/mL, 3 mg/mL, 1 mg/mL, 0.5 mg/mL). The samples were prepared and parameters were set up as described above. The H7-767 diffusion coefficients as a function of these protein concentrations were measured by DLS. The diffusion interaction parameter (kD) was determined by a linear fit of the measured diffusion coefficients (Dₘ) as a function of protein concentration (C) using the equation as follows:

Dₘ=D₀(1+kD*C) where D₀ is the self-diffusion coefficient obtained from (diffusion coefficient as C→0).

The formulations at kD values <−8 mL/g demonstrated strong repulsive molecular interactions; kD values in the range −1 to −5 mL/g depicted low repulsive interactions, and kD at >−8 mL/g showed strong attractive molecular interactions.

Determination of temperature of aggregation of H7-767 (Tagg)—The H7-767 samples were prepared as described above; additionally, ˜20 μL of mineral oil was added over each sample well in the plate to prevent evaporation during temperature ramping. Most parameters were set up as described above; the changes being the experiment set up as discrete temperature increments from 25° C. to 75° C. at 2° C. temperature increments for 5 acquisitions at 10 seconds of acquisition time. The molecule's Tₒₙₛₑₜ was determined as the onset temperature of thermal transition and aggregation temperature (Tagg) was determined based on the temperature at which a significant increase in the Rh and PD was observed with obvious aggregate formation.

### DSC (Differential Scanning Calorimetry)

To determine the midpoint transition temperature of unfolding (Tm) of different domains of H7-767 molecule and onset temperature (Tₒₙₛₑₜ) of unfolding, MicroCal VP-Capillary DSC (Malvern) using 2.5 mL, 22-gauge CTC syringe (ThermoFisher, NC9636112) with software 2.0 version was employed. 1 mg/mL of formulation sample at 800 μL alongside respective formulation buffer was loaded to the 1.0 mL 96-well deep well plates (NEST, 502101) and covered with silicone cover (ThermoFisher, NC0875398). A pair of respective formulation buffers was used to obtain the baseline for each H7-767 formulation. The samples were subjected to thermal ramping from 25° C. to 95° C. with a scan rate of 60° C./hr with a filtering period of 10 seconds without rescan or feedback mode to obtain unique scans. The prescan thermostat was set at 7 minutes at 25° C. and post-cycle thermostat at 25° C.

For data analysis, the exact concentration (M) of the H7-767 sample was applied followed by linear baseline subtraction (fit manually as needed). To fit multiple thermodynamic transitions, the “non-2-state: cursor init” option was used to select transitions of three domains (CH2, Fab, and CH3); the fourth transition as an intermediate transition was employed at the Fab domain. A non-linear curve fitting was carried out to iterate the fit using “200 Liter” function until the chi² value was not reduced any further. The information on Tₒₙₛₑₜ (the initial unfolding transition), the midpoint of transition of thermal unfolding of each domain (Tm), and their enthalpies were obtained. Typically, the higher the Tₒₙₛₑₜ and Tm, the more stable the molecule is in the formulation.

### SE-HPLC (Size Exclusion-High Performance Liquid Chromatography)

To determine the purity of the molecule based upon monomer %, high molecular weight species (HMWS %) and low molecular weight species (LMWS %), Size Exclusion-High Performance Chromatography (SE-HPLC; Waters) equipped with UV detector set for 280 nm absorbance detection, HPLC column (TOSOH TSKgel G3000SWx1, 08541), and guard column (TOSOH TSKgel Guard SWx1, 08543) was used. The mobile phase used was 50 mM sodium phosphate (Acros Organics, 271750010), and 450 mM L-arginine hydrochloride (EMD Millipore, 181003) at pH 7.0. A duplicate of the gel filtration standard (BioRad, 151-1901) flanking the formulation samples was used for system suitability and a triplicate of a reference standard were used for sample suitability test. Sample injection volume was set such that 150 μg of a sample was injected for each chromatogram with 35 minutes run time at 0.5 mL/min flow rate for isocratic elution.

The H7-767 percentage area of HMWS, Monomer, and LMWS were calculated as below:

$$Area ( \% ) = \frac{Area of Peak}{Total peak area} * 100 \%$$

### PEG (Polyethylene Glycol Solubility Assay)

To determine the solubility of the molecule in different formulations based on the PEG precipitation assay, the A₃₅₀ for 1 mg/mL H7-767 was measured at increasing concentrations of PEG-4000 at (0%, 2%, 4%, 6%, 8%, 10%, 12%, 14%, and 20%). 100 μL of the sample per well was placed in 96-well clear bottom microplate (Corning; 4850) in triplicates and incubated for 15 minutes after preparation. The absorbance measurements were acquired at the wavelength of 350 nm after the microplate was loaded to SpectraMax M3 multi-mode microplate reader. The A₃₅₀ measurement of each formulation buffer was subtracted from respective sample's measurement. The average values of the A₃₅₀ for 1 mg/mL H7-767 formulations were used for analysis.

icIEF (Imaged Capillary Isoelectric Focusing)

To evaluate the charge heterogeneity of the molecule as main peak %, acidic species %, and basic species % and to determine the experimental isoelectric point (pI), icIEF was employed. A 180 μL of master mix solution per sample containing 27.8% of 8M urea solution (Fisher; U15-500), 38.9% of 1% methyl cellulose (Protein Simple; 101876), 4.4% of pharmalyte pH 3-10 (Cytiva; 17-0456-01), 1.1% of pI marker 6.14 (Protein Simple; 102220), 1.1% of pI marker 9.50 (Protein Simple; 101996), and 26.7% of MilliQ water was prepared. 20 μL of H7-767 sample diluted to 4 mg/mL was added to the 180 μL of master mix solution. A blank solution was prepared by combining 20 μL of MilliQ water with 180 μL of master mix solution. The samples were vortexed for 30 seconds at high speed and spun down at 13000 RPM for 3 minutes. The system suitability solution was prepared by mixing 160 μL of system suitability test mix (Protein Simple; 046-044) to 40 μL of water in a centrifuge tube, vortexed 3 times, and centrifuged at 10,000 g for 3 minutes to sediment any particulates. Without generating any bubble, 180 μL of the preparation solution was transferred to a 96-well plate (Protein Simple; 046-021); the plate was centrifuged at 1000 g for 5 minutes and loaded to Maurice icIEF instrument (Protein Simple; 092-002). The reagents-0.5% methyl cellulose (Protein Simple; 102505); Fluorescence calibration standard (Protein Simple; 046-025); vials with water; and empty vial were placed on instrument according to manufacturer's instructions.

The icIEF cartridge (Protein Simple; NC1085630) was loaded with 2 mL of catholyte solution in the catholyte tank and 2 mL of anolyte solution in anolyte tank using the electrolyte kit (Protein Simple; 102506). The experiment set up implemented focusing period I of 1 minute at 1500 V, focusing period II of 6 minutes at 3000 V, detection with 5 exposures, and pI markers selected as 6.14 and 9.50. The electropherograms were analyzed using Maurice icIEF software and integration using Empower 2 to obtain main species, acidic species and basic species.

The H7-767 isoelectric point (pI) was calculated as below:

$$Average pI ( \% ) = \frac{Sum of all pI values for reference samples}{Number of injections} * 100 \%$$

### CGE (Capillary Gel Electrophoresis)

The Sciex PA80 with PDA detector with 100×200 aperture (Sciex; 144712) was used for the experimental analysis of reduced and non-reduced CGE.

The reducing master mix contained 91.8% SDS-MW sample buffer (Sciex; A30341), 2.4% of 10 kDa internal standard (Sciex; A26487) and 5.9% of 2-mercaptoethanol (Sigma; M7154). The non-reducing master mix contained 91.8% of 1% SDS (Sciex; AM9822), 5.9% of 250 mM N-Ethylmaleimide (Thermo Scientific; 23030), and 2.4% of 10 kDa internal standard (Sciex; A26487). To 15 μL of H7-767 formulation sample, 180 μL of respective master mix for reducing and non-reducing conditions were added. The non-reducing samples were heated at 65° C. for 10 minutes. After equilibration of all samples to room temperature, 85 μL of each sample was transferred to 200 μL micro vials (Sciex; 144709). Bubbles were removed by centrifugation of vials at 500 g for 1 minute. The instrumental parameters were set at 60 Hz, negative polarity, sample rate at 2, scale factor of 1 using only channel 1, apex detection of 13.0-38.0 min, peak width of 30, detection threshold of 100, minimum area of 1000, and minimum peak height of 100.

The total time corrected area (TCA) for each peak is calculated as:

Total TCA=Sum of TCAs for all peaks migrating after the 10 kDa internal standard

### For Non-Reduced (NR) Samples

Purity % (non-reduced) for each peak is calculated as:

$$Purity \% ( NR ) = \frac{TCA of IgG}{Total TCA} * 100 \%$$

Heavy Chain attached to Heavy Chain-Light Chain halfmer (HHL) % (non-reduced) for each peak is calculated as:

$$HHL \% ( NR ) = \frac{TCA of HHL}{Total TCA} * 100 \%$$

Fragments % (non-reduced) for each peak is calculated as:

$$Fragments \% ( NR ) = \frac{Sum of TCAs for all peaks migrating before the HHL Peak}{Total TCA} * 100 \%$$

### For Reduced Samples

Purity % (reduced) for each peak is calculated as:

$$Purity \% ( R ) = \frac{TCA of heavy chain ( HC ) + TCA of light chain ( LC )}{Total TCA} * 100 \%$$

Non-glycosylated heavy chain (NGHC) % (reduced) for each peak is calculated as:

$$NGHC \% ( R ) = \frac{TCA of NGHC}{TCA of NGHC + TCA of heavy chain} * 100 \%$$

Fragments % (reduced) for each peak is calculated as:

$$Fragments \% ( R ) = \frac{Sum of TCA for all non - LC / NGHC peaks migrating before HC}{Total TCA} * 100 \%$$

### Relative Potency by Cell-Based Potency Assay

The relative potency (RP) of H7-767 was determined by selective IL-2 stimulation of PD-1/IL-2R cells. The PD-1/IL-2R cell line was transfected to express the human IL-2Rβ receptor subunit, and lentivirally transduced to express human PD-1. This bioassay determines the proliferation of PD-1/IL-2R cells by measuring luminescence to quantify adenosine triphosphate (ATP) in PD-1/IL-2R cell culture, which is directly proportional to number of cells in the culture.

Using a 7.5% BSA solution (Gibco; 15260-037), 0.1% BSA was prepared in sterile cell culture grade water (Corning; 25-055-CM) and filtered with 0.2 μm filter unit on the day of preparation. A working stock of 10 μg/mL (645 nM) human IL-2 was prepared adding 5 mL of 0.1 mg/mL of human IL-2 stock (PeproTech; AF20002 500UG) to 45 mL 0.1% BSA. The assay media was prepared by using 860 mL of RPMI 1640 with glutamine (Corning; 10-040-CM), 100 mL of Heat Inactivated Fetal Bovine Serum (Corning; 35-011-CV), 10 mL of 100× Glutamax (Gibco; 35050-061), 10 mL of 100× Penicillin/Streptomycin (Gibco; 15140-122), and 20 mL of 50 mg/mL Hygromycin (Invitrogen; 10687010). The working stock of 10 μg/mL human IL-2 was prepared; the working stock at 1:1000 was added to assay media resulting in 10 ng/ml final concentration of human IL-2. If cells passage was performed, the target seeding density (viable cells/mL) of approximately 4000 was obtained by monitoring the cell growth such that cells were in a log phase between 3-5 days.

The assay ready PD-1/IL-2R cells were resuspended in 20-30 mL assay media followed by cell count using Nucleo Counter (Chemometec; NC-3000) or Hemocytometer (Fisher; 02-671-5) and the viability was >80%. The cells in assay media at 1±0.2×10⁵ cell/mL suspension was prepared. Next, 50 μL of the cell suspension was used for each well of the plate (Greiner Bio-One; 655083) to equal 4-6×10³ cells/well and incubated for 64-72 hours in the 37±2° C., 5±2% CO2, ≥75% humidity incubator (Thermo; Hera Cell 240). On assay day 4, these assay plates were equilibrated at room temperature, 100 μL of CellTiter-Glo®2.0 reagent (Promega; G9242) was added to each well, plate was incubated for 10-60 minutes avoiding light and luminescence was measured using a luminescence plate reader (Molecular Devices; SpectraMax L Config) with glow-type luminescence read capabilities with a target wavelength of 470 nm and integration time of 1 second per well.

A well-characterized representation lot of H7-767 was used as an internal control (IC) (DS not intended for clinical use) and a reference stock (RS) at final starting concentration of 2000 ng/ml with a series of dilutions to (740 ng/ml, 274 ng/ml, 101 ng/ml, 38 ng/mL, 14 ng/mL, 5.2 ng/mL, 0.7 ng/mL, and 0.3 ng/mL) were used. SoftMax Pro GxP software (V.7.0; Molecular Devices) was used to perform 4-parameter logistic curve fitting of the data and assess curve parallelism by F-test to calculate the Half Maximal Effective Concentration (EC₅₀) values of reference stock (RS), internal control (IC), and test sample (TS) and the Relative Potency (RP) of IC and TS.

$$Relative potency ( RP ) \% = \frac{EC 50 ( reference )}{EC 50 ( test )} * 100 \%$$

SoftMax Pro software was used to calculate the asymptote ratio by dividing the upper asymptote of the unconstrained RS curve by the mean relative light units (RLU) of wells containing media+ cells. The reportable value (RV) of a test sample is the arithmetic mean of the RP from two independent assay runs using independent dilutions of reference standard and test sample and independently prepared cells and different plate formats. The coefficient of variation (CV) of two plates and the reportable value (RV) of each test sample were calculated as follows:

$$Reportable Value ( RV ) = \frac{RP 1 \% + RP 2 \%}{2}$$

$$Coefficient of variation ( CV ) = \frac{Standard Deviation ( sample )}{RV} * 100 \%$$

### Protein Concentration Measurement by SoloVPE

The protein concentration of H7-767 was measured using SoloVPE system (CTech™/Repligen) equipped with spectrophotometer (Cary-60 of Agilent). The average of three repeats was used as the protein concentration of the H7-767 formulation.

### Turbidity by Turbidimeter

The degree of opalescence of H7-767 was measured using HACH® TL2300 Tungsten Lamp Turbidimeter (HACH; LPV444.99.00210). Turbidity measurement was determined as the average of three replicates of the same sample.

### pH Measurement by pH Meter

The pH of H7-767 samples were measured using pH benchtop meter (Mettler Toledo™; 01-914-325) and Micro Pro-ISM Electrode (Mettler Toledo™; 51344163). The reportable pH value was determined as an average of three replicates of the same sample.

### Osmolality by Osmometer

The osmolality of H7-767 sample was measured using an OsmoPro® osmometer (Advanced Instruments) with evaporation cover (Advanced Instruments; 112081) and turntable (Advanced Instruments; 112077). Osmolality measurements were obtained for three replicates of a H7-767 sample and averaged.

### Sub-Visible Particle Count by Microflow Imaging (MFI)

The sub-visible particles count was measured using microflow imaging (Protein Simple; MFI 5000 Series with Bot1) set up with 1000 μL disposable tips (Tecan; 30000631). A 96-deepwell plate white border (Fisher; P-0000068-00) was thoroughly rinsed with MilliQ water before adding 2 mL of water to initial two wells and 1.1 mL of H7-767 samples in triplicates. The experiment run was set up such that the test samples were flanked with flush and dry tests using 0.9 mL of water at 6 mL/min flow rate for 4 times and an illumination optimized with 0.22 mL of water. A successful baseline using MilliQ water was established at particle count of less than 300 particles/mL measured with ECD between 2 and 300 microns. This procedure was repeated after each sample injection through the flow cell. The particle count reported as concentration (#/mL) in the specified ranges were differential particles/mL. The cumulative particles/mL was determined as the sum of particles between ≥1 micron and ≤300 micron for ≥1 micron; between ≥2 micron to ≤300 micron for ≥2 micron, between ≥5 micron to ≤300 micron for ≥5 micron; between ≥10 micron to ≤300 micron for ≥10 micron; and between ≥25 micron to ≤300 micron for ≥25 micron.

### Buffer and pH Study

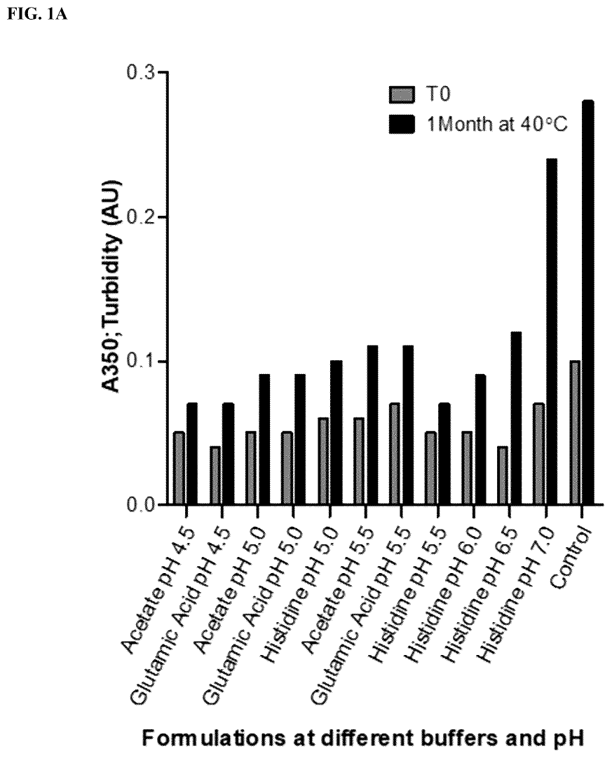

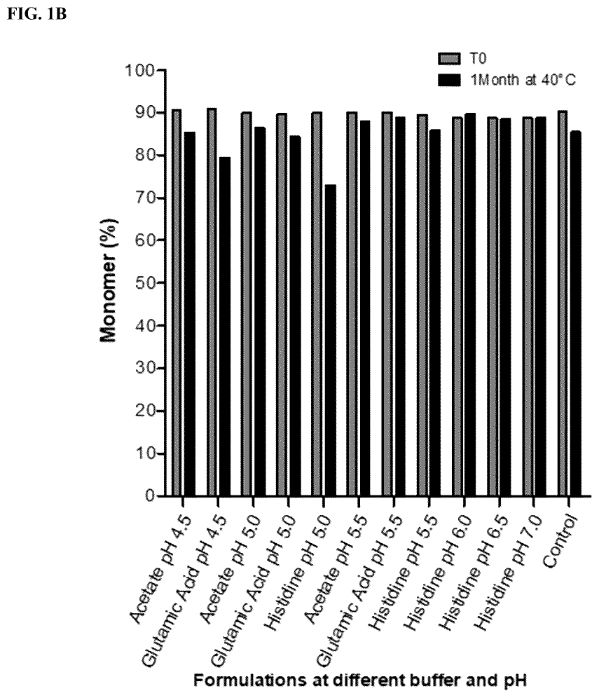

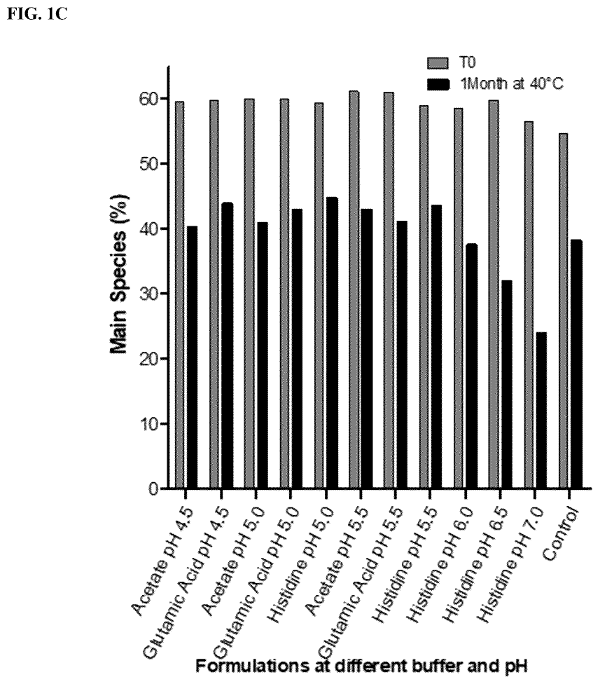

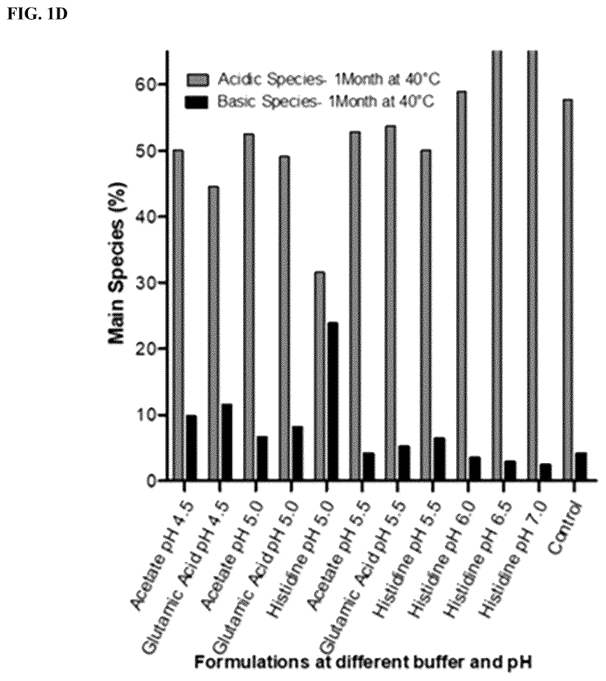

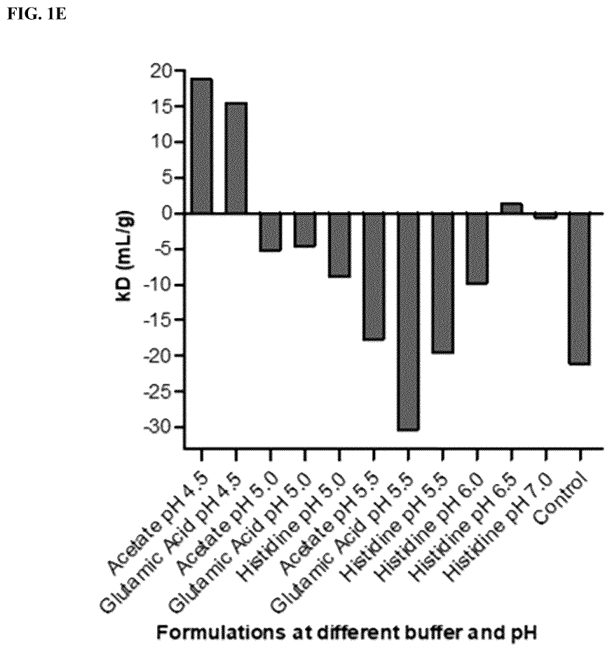

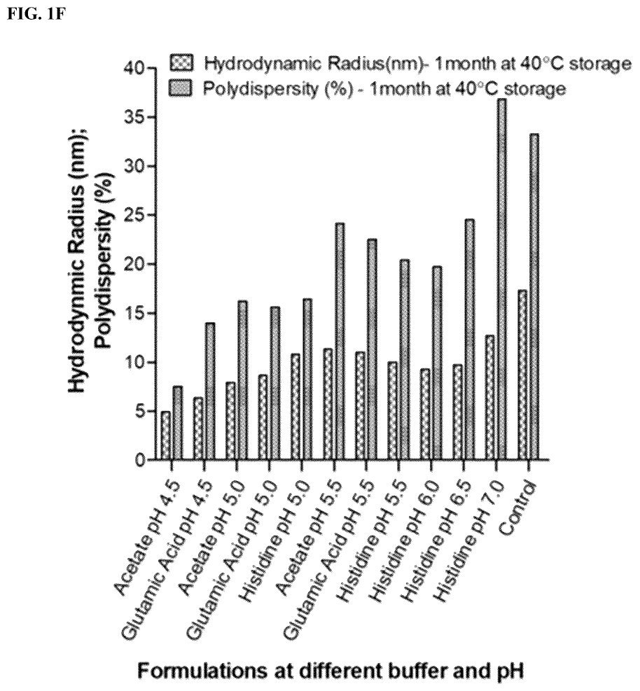

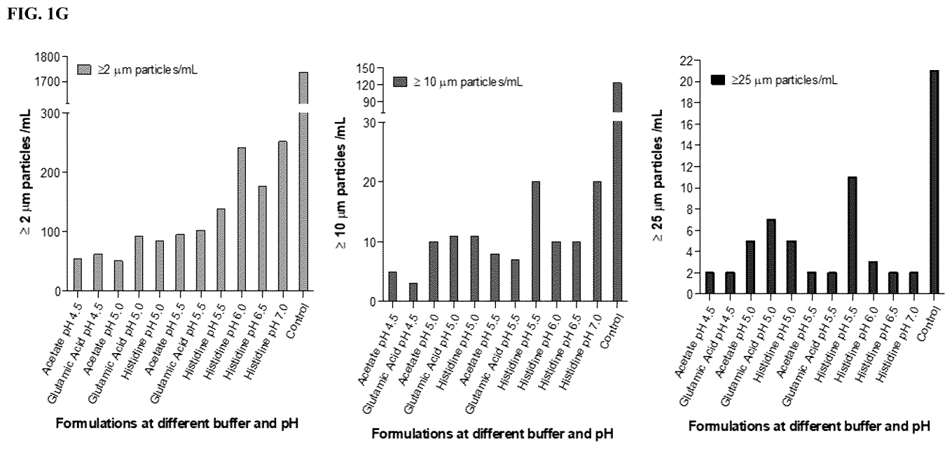

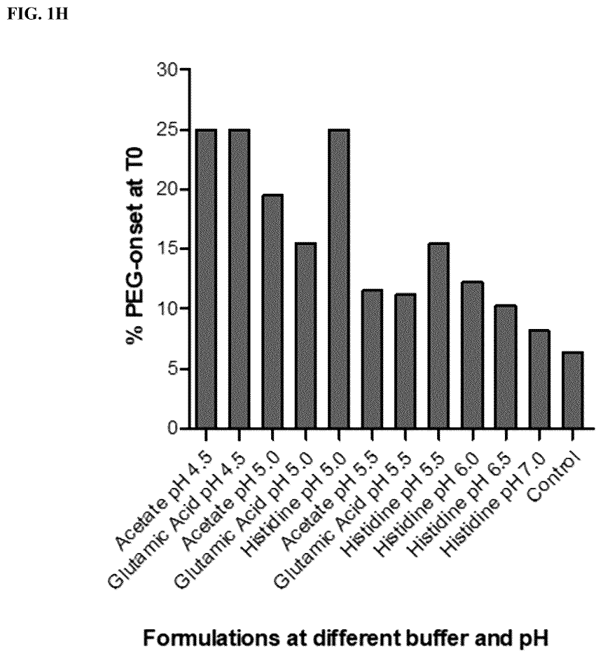

Initial screening studies were conducted to determine the optimal buffer and pH conditions for 10 mg/mL liquid H7-767 formulation using three different buffer systems—acetate (pH 4.5, 5.0, 5.5), glutamic acid (pH 4.5, 5.0, 5.5), and histidine (pH 5.0, 5.5, 6.0, 6.5, 7.0). Equimolar arginine/glutamate in histidine at pH 6.0 was used as the control sample. All formulations also contained polysorbate 80 (PS80) at 0.05%. Surfactant was necessary as H7-767 forms high amounts of visible particles and fibrils that interfered with most testing and handling. Formulations were examined at TO and 1 month at 40° C. storage as a stress condition. The H7-767 control formulation with 100 mM arginine/glutamate in 25 mM histidine and 0.05% PS80 at pH 6 showed proteinaceous visible particles and the highest turbidity (FIG. 1A, FIG. 1B, FIG. 1C, FIG. 1D, FIG. 1E, FIG. 1F, FIG. 1G, FIG. 1H, and FIG. 1I and Table 1), significant attractive protein-protein interactions with reduced colloidal stability (FIG. 1E and FIG. 1F), aggregate formation (FIG. 1B), and the least solubility (FIG. 1D), which is undesirable for a drug product. The appearance and turbidimetry measurements at A350 of each formulation is summarized in Table 1.

TABLE 1
Visual inspection and turbidimetric
measurements show effect of stress.

| | Visual inspection | | Absorbance (A350) | |
|---|---|---|---|---|
| | | 1 Month | | 1 Month |
| Formulation | T0 | at 40° C. | T0 | at 40° C. |
| Acetate pH 4.5 | C | C | 0.05 | 0.07 |
| Glutamic Acid pH 4.5 | C | C | 0.05 | 0.09 |
| Acetate pH 5.0 | S | S | 0.06 | 0.11 |
| Glutamic Acid pH 5.0 | C | S | 0.04 | 0.07 |
| Histidine pH 5.0 | S | S | 0.05 | 0.09 |
| Acetate pH 5.5 | S | O | 0.07 | 0.11 |
| Glutamic Acid pH 5.5 | S | O | 0.06 | 0.10 |
| Histidine pH 5.5 | S | O | 0.05 | 0.07 |
| Histidine pH 6.0 | S | S | 0.05 | 0.09 |
| Histidine pH 6.5 | S | S | 0.04 | 0.12 |
| Histidine pH 7.0 | S | HO | 0.07 | 0.24 |
| Control | O | CL | 0.10 | 0.28 |

T0: initial time point; 1M40C: after 1 month storage at 40° C.; C: clear; CL: cloudy; O: opalescent; S: slightly opalescent; HO: highly opalescent; O: opalescent.

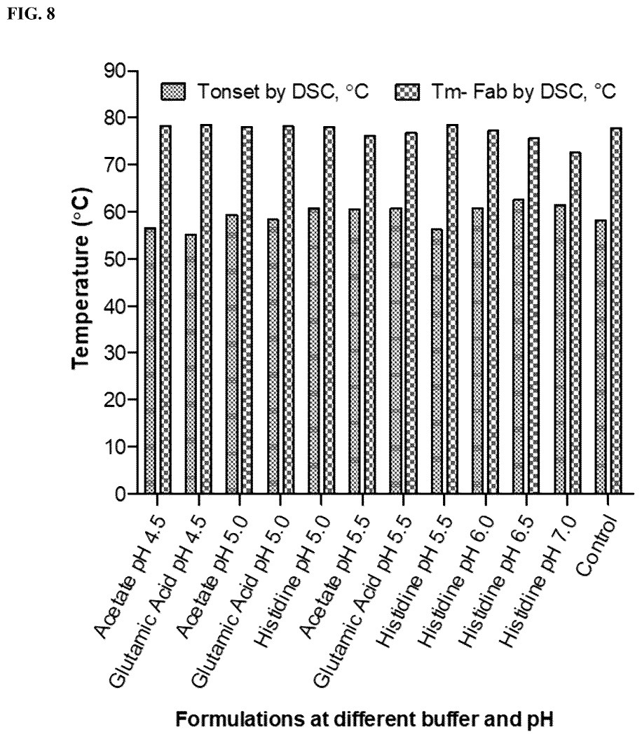

Solubility by PEG precipitation assay indicated that formulations in acetate and glutamate at pH 4.5, and also histidine at pH 5.0, had similar highest solubility. Once pH increased, the solubility decreased for all buffer systems with the least soluble being the control formulation, followed by histidine formulations at pH 7.0 and 6.5 (FIG. 1H). Turbidities for all formulations were similar at initial testing, except for the control sample, though it increased upon storage at 40° C. Control formulation and formulation in histidine at pH 7.0 had the highest turbidity. Acetate and glutamate at pH 5.5 and histidine at pH 6.5 and 5.0 had relatively higher turbidities compared to other samples (FIG. 1A). Formulations with histidine at pH>6.0 also showed reduced enthalpy by DSC that indicated loose H7-767 structure with disrupted bonds (FIG. 1I, FIG. 8, and Table 2).

TABLE 2
Enthalpies of unfolding of H7-767
in different buffer and pH systems.

| | Enthalpy of Unfolding ΔH |
|---|---|
| Acetate pH 4.5 | 945860 |
| Glutamic Acid pH 4.5 | 914940 |
| Acetate pH 5.0 | 1043100 |
| Glutamic Acid pH 5.0 | 972380 |
| Histidine pH 5.0 | 977850 |
| Acetate pH 5.5 | 1130840 |
| Glutamic Acid pH 5.5 | 1178800 |
| Histidine pH 5.5 | 1037090 |
| Histidine pH 6.0 | 895100 |
| Histidine pH 6.5 | 838300 |
| Histidine pH 7.0 | 670840 |
| Control | 923200 |

Stressed conditions at 40° C. significantly affected H7-767 by increasing the sub-visible particles for the control formulation at pH 6.0 and the histidine formulation at pH 7.0 (Table 3 and FIG. 1G).

TABLE 3
Concentration of sub-visible particulate per mL by MFI in
various formulations after storage for 1 month at 40° C.

| | Concentration of sub-visible particles (#/mL) | | | | |
|---|---|---|---|---|---|
| | after 1 month at 40° C. | | | | |
| Formulation | ≥1 μm | ≥2 μm | ≥5 μm | ≥10 μm | ≥25 μm |
| Acetate pH 4.5 | 4055 | 876 | 149 | 16 | 3 |
| Glutamic | 4491 | 1258 | 472 | 206 | 64 |
| Acid pH 4.5 | | | | | |
| Acetate pH 5.0 | 23181 | 5566 | 1230 | 187 | 10 |
| Glutamic | 8527 | 1765 | 498 | 171 | 53 |
| Acid pH 5.0 | | | | | |
| Histidine pH 5.0 | 10979 | 2009 | 571 | 184 | 51 |
| Acetate pH 5.5 | 16535 | 3651 | 909 | 203 | 15 |
| Glutamic | 17941 | 6115 | 2094 | 762 | 147 |
| Acid pH 5.5 | | | | | |
| Histidine pH 5.5 | 9156 | 1489 | 256 | 51 | 7 |
| Histidine pH 6.0 | 8345 | 1363 | 263 | 72 | 31 |
| Histidine pH 6.5 | 15535 | 2945 | 540 | 131 | 21 |
| Histidine pH 7.0 | 481260 | 103552 | 22442 | 5381 | 457 |
| Control | 12629594 | 2468778 | 491811 | 44840 | 123 |

Increased aggregation propensity by DLS for acetate and glutamate formulations with pH>5.0 and histidine formulations with pH>6.0 are shown in FIG. 1F.

High soluble aggregate formation by SE-HPLC in histidine formulation at pH<5.5 and glutamate formulation at pH<5.5 are shown in FIG. 1B. Charge heterogeneity was also affected in acetate and glutamate buffers, but histidine formulations at pH<5.5 showed a significant increase in basic species, while at pH>5.5 showed a significant increase in acidic species; the same was observed for the control formulation (FIG. 1C and FIG. 1D).

Global ranking by risk-based assessment of H7-767 quality attributes for optimal buffer and pH selection indicated acetate buffer at pH 5.0 to be the optimal buffer condition for H7-767.

### Analysis of Formulations Containing Surfactants and Formulation Containing Sucrose

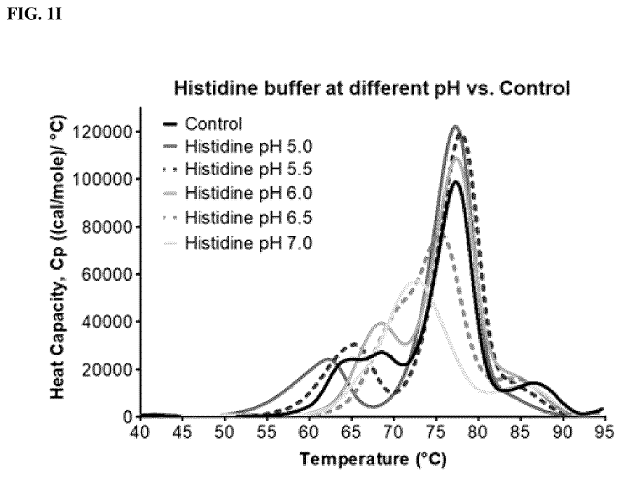

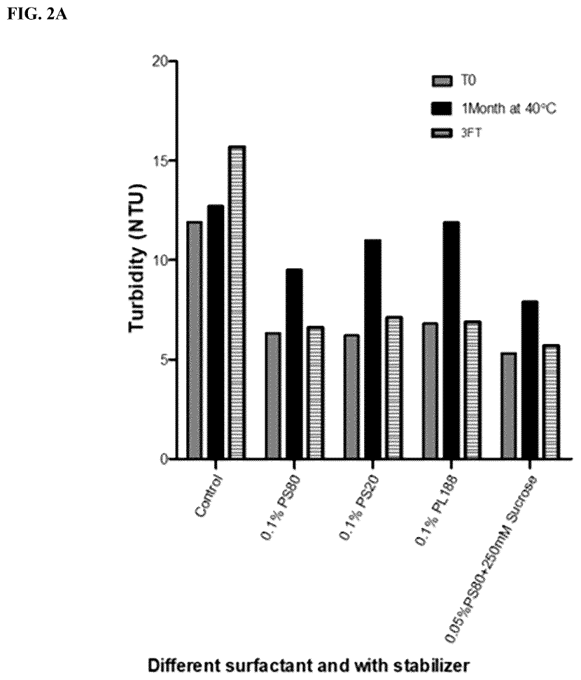

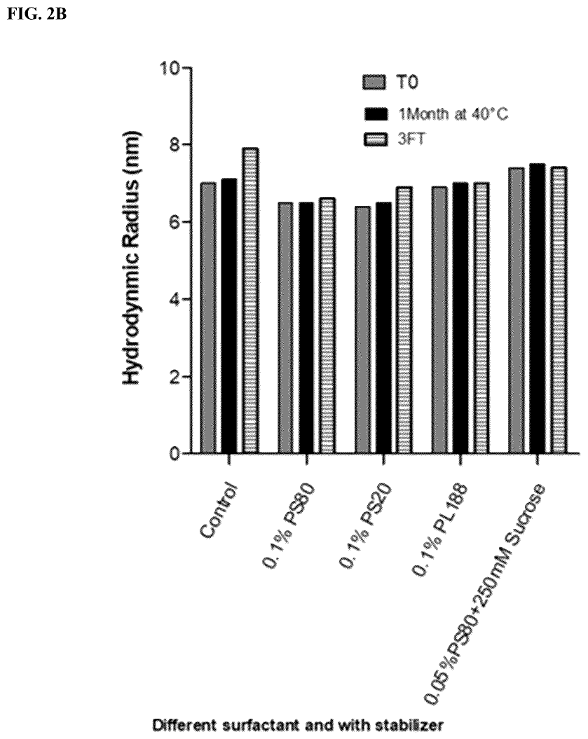

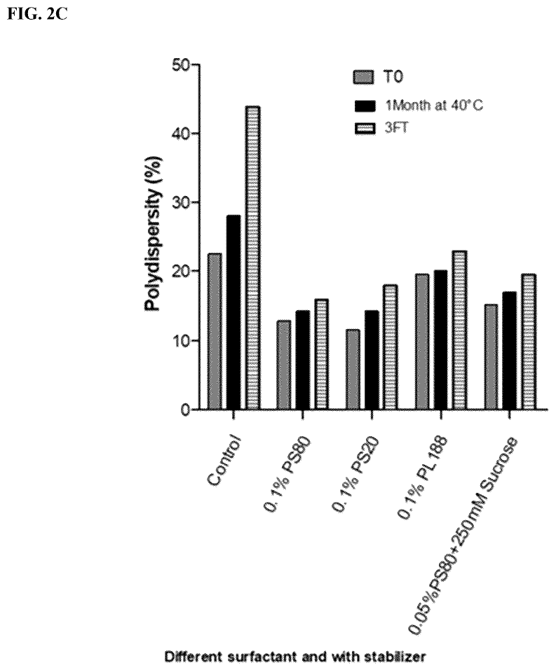

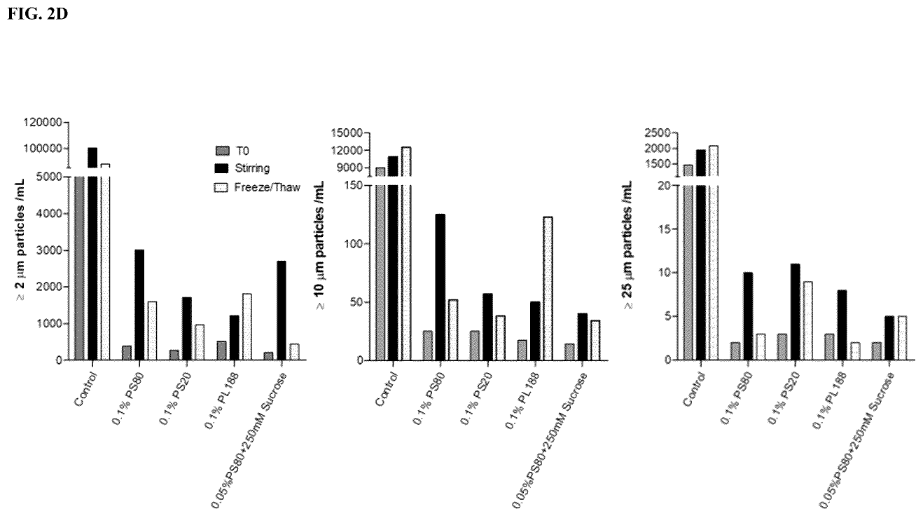

The physiochemical properties of H7-767 were evaluated in 20 mM acetate at pH 5.0, in the presence of different surfactants such as polysorbate 80 (PS80), polysorbate 20 (PS20), and poloxamer 188 (PL188) at 0.1% level (FIG. 2A, FIG. 2B, FIG. 2C, and FIG. 2D). Control formulation was H7-767 in acetate buffer at pH 5.0 without any surfactant. Formulations were analyzed at TO, and after freeze-thaw cycling and stirring (stressed conditions). Most formulations had visible particles at TO or after stressed conditions. The control sample generated the greatest number of visible particles and fibrils and sub-visible particles with overall low colloidal stability (FIG. 2D). The PL188 formulation also showed aggregate formation by DLS (FIG. 2C). Overall, PS80 and PS20 under stress conditions of stirring and 3 freeze-thaw cycles were the most promising surfactants based on the lowest hydrodynamic radius and polydispersity by DLS (FIG. 2B and FIG. 2C), and least turbidity measured by turbidimeter (FIG. 2A).

A formulation of 10 mg/mL H7-767, 20 mM acetate, 250 mM sucrose, and 0.05% PS80 at pH 5.0 were evaluated. The addition of sucrose showed a slight improvement in visible particulate formation. However, this formulation started to form visible particles during longer storage at 2-8° C., or when in contact with different materials when handling the molecule. Improvement in colloidal and thermal stability with higher purity was still required for these formulations. Comparing all the formulations, it was noted that 0.1% of PS80 did not have any benefits compared to formulations with 0.05% PS80 and sucrose; the latter performed better in most instances suggesting that H7-767 requires PS80 at a concentration of at least 0.05% (FIG. 2A, FIG. 2B, FIG. 2C, and FIG. 2D).

### Excipient Evaluation

The protein-protein modifiers imidazole, arginine, histidine, sodium chloride (NaCl), and glycine were evaluated at varying concentrations in the range of 100 mM to 500 mM in formulations containing 10 mg/mL H7-767, 20 mM acetate, 250 mM sucrose, and 0.05% PS80, pH 5.0.

H7-767 in arginine formulations at 100 mM, 200 mM, 300 mM, and 500 mM, and histidine formulations at 100 mM and 200 mM all demonstrated slightly opalescent solutions with multiple visible particles, especially at higher concentrations (data not shown).

H7-767 in NaCl formulations at 100 mM, 300 mM, 600 mM, and 800 mM, and imidazole formulations at 150 mM, 300 mM, and 500 mM exhibited highly opalescent, cloudy solutions with protein precipitation and protein coagulation on the top surface (data not shown).

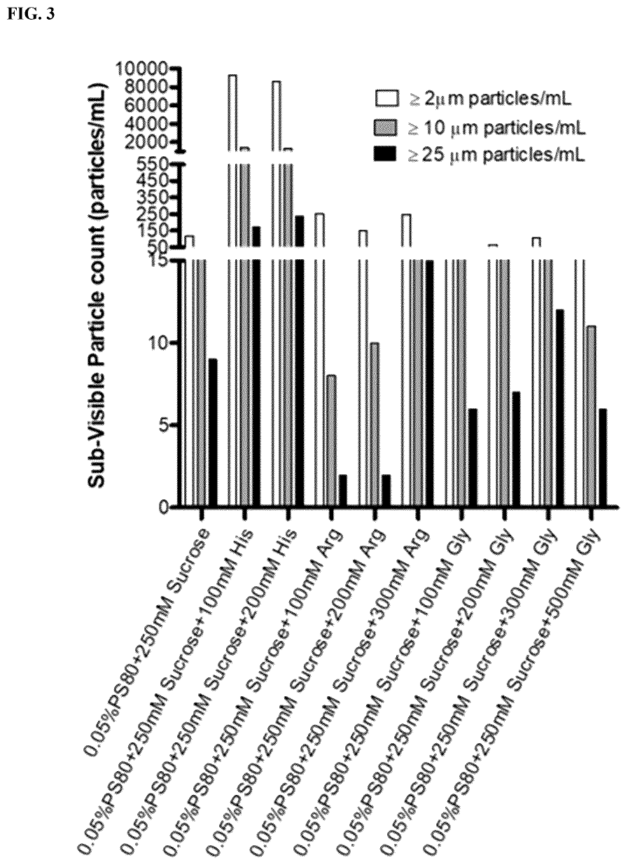

In contrast, glycine formulations at 100 mM, 200 mM, 300 mM, 400 mM, and 500 mM all improved the overall colloidal stability of the molecule and demonstrated slightly opalescent solutions that were free of visible particles during visual inspection (FIG. 3).

To evaluate the effects of different protein concentrations, H7-767 was evaluated at a concentration range of 2 mg/mL to 10 mg/mL in formulations containing polysorbate 20 at 0.1% or polysorbate 80 at 0.05% to 0.1%, sucrose at 200 mM to 250 mM, and glycine at 100 mM to 400 mM. Specifically, additional studies were performed with:
H7-767 at 5 mg/mL and 2 mg/mL in 20 mM acetate at pH 5.0, with 0.1% PS20;
H7-767 at 5 mg/mL and 2 mg/mL in 20 mM acetate at pH 5.0, with 0.1% PS80;
H7-767 at 5 mg/mL and 2 mg/mL in 20 mM acetate at pH 5.0, with 0.05% PS80, and 250 mM sucrose;
H7-767 at 10 mg/mL in 20 mM acetate at pH 5.0, with 0.05% PS80, 200 mM sucrose, and 100 mM glycine; and
H7-767 at 10 mg/mL in 20 mM acetate at pH 5.0, with 0.05% PS80, 200 mM sucrose, and 400 mM glycine.

To evaluate the performance of these H7-767 formulations upon stress conditions, the samples in 10R glass vials were subjected to either stirring for 20, 30, or 60 minutes at 150 RPM at room temperature under normal light condition, or three freeze/thaw cycles (3 FT): freezing at −70° C. for at least 6 hours (until completely frozen) followed by thawing at 2-8° C. for not less than 4 hours.

Most samples started to form white visible particles within a minute once transferred from 10R glass vial to a glass tube for turbidity measurements, indicating the sensitivity of H7-767 to shear stress and/or to different contact materials. The most opalescent samples with the highest amount of visible particles were the samples at 2 mg/mL without any stabilizers (sucrose or glycine) indicating that surfactant only was not enough to protect the protein from shear stress and interface-mediated instabilities. The sample containing 200 mM sucrose with 100 mM glycine formed just a few visible particles. The sample containing 400 mM glycine showed no visible particles. Similar observations were made once formulations were left in glass vials at 2-8° C. for 24 hrs—most formulations formed visible particles that differed in size and quantity throughout the samples, except formulation with 200 mM sucrose and 400 mM glycine (data not shown).

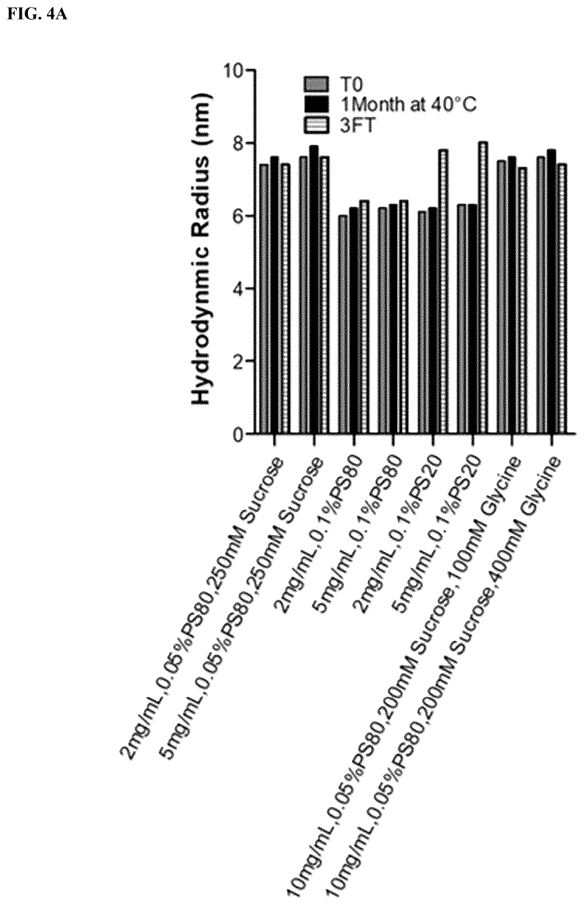

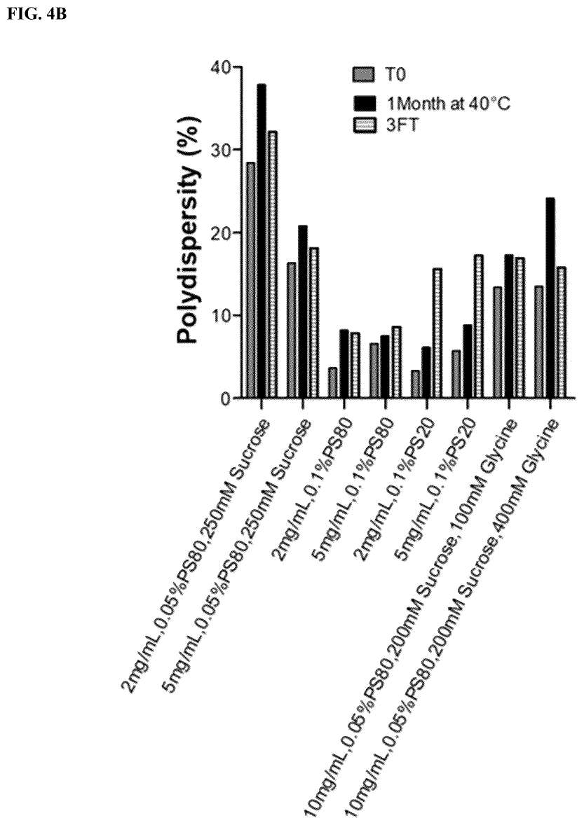

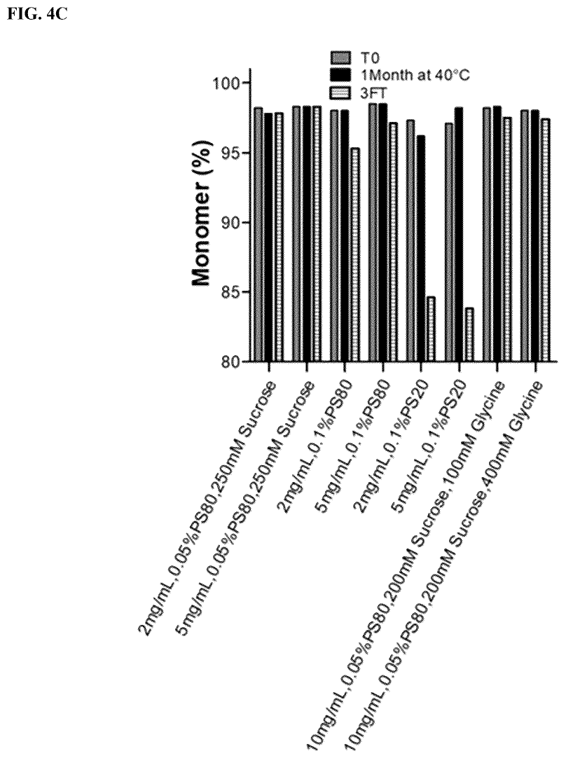

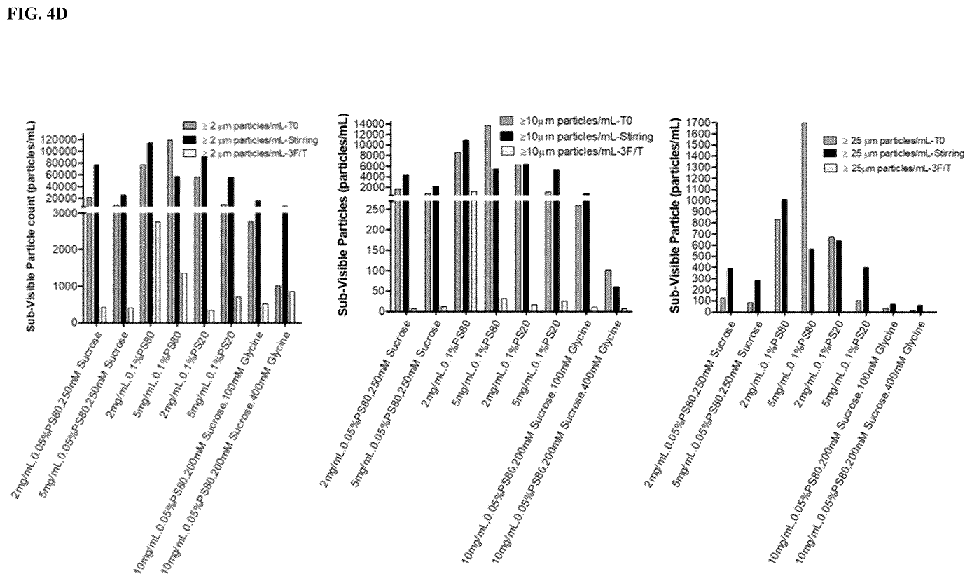

Hydrodynamic radius (Rh) was observed in the 6.1-8.0 nm range for formulations with PS80 and PS20, compared to Rh within 7.3-7.8 nm for formulations with stabilizers (sucrose and glycine), which suggests an additional layer added to the H7-767 molecule by those stabilizers that act as preferential exclusion excipients and modify the water-excipient layer around the molecule (FIG. 4A). Polydispersity measurements (FIG. 4B) indicated the propensity of aggregation in these formulations and was highest in formulations having 2 mg/mL H7-767, 20 mM acetate at pH 5.0, with 0.05% PS80, and 250 mM sucrose. High polydispersity was also observed in PS20 formulations after 3 FT cycles and for 400 mM glycine after 60 minutes of stirring. Nevertheless, formulations with 100 mM and 400 mM glycine formed the least sub-visible particles compared to all other formulations (FIG. 4D).

SE-HPLC analysis, which monitored purity, showed that freeze-thaw (FT) stress caused most of the loss in monomer (i.e., the single, properly folded unit of the immunoconjugate without any association with other immunoconjugate molecules or aggregation) for all formulations-monomer for formulations with PS20 dropped to ˜84% (Table 4), with an increase in aggregates. It was also observed that formulations at 2 mg/mL were more susceptible to degradation (FIG. 4C).

TABLE 4
Purity of H7-767 at initial timepoint and stressed conditions
at different protein, surfactant, and excipient concentrations.

| | | H7-767 formulations | | | | | | | |
|---|---|---|---|---|---|---|---|---|---|
| | | | | | | | | 10 | 10 |
| | | | | | | | | mg/mL, | mg/mL, |
| | | 2 | 5 | | | | | 0.05% | 0.05% |
| | | mg/mL, | mg/mL, | | | | | PS80, | PS80, |
| | | 250 mM | 250 mM | 2 | 5 | 2 | 5 | 200 mM | 200 mM |
| | | sucrose/ | sucrose/ | mg/mL, | mg/mL, | mg/mL, | mg/mL, | sucrose, | sucrose, |
| Quality | | 0.05% | 0.05% | 0.1% | 0.1% | 0.1% | 0.1% | 100 mM | 400 mM |
| attribute | Conditions | PS80 | PS80 | PS80 | PS80 | PS20 | PS20 | Glycine | Glycine |
| Monomer, % | T0 | 98.2 | 98.3 | 98.0 | 98.5 | 97.3 | 97.1 | 98.2 | 98.0 |
| | 1 FT | 97.8 | 98.1 | 97.3 | 98.1 | 92.8 | 92.6 | 97.3 | 97.4 |
| | 3 FT | 97.8 | 98.3 | 95.3 | 97.1 | 84.6 | 83.8 | 97.5 | 97.4 |
| Dimer, % | T0 | 1.0 | 0.9 | 1.2 | 0.9 | 1.3 | 1.5 | 0.8 | 1.0 |
| | 1 FT | 1.3 | 1.0 | 1.7 | 1.2 | 2.8 | 2.6 | 1.2 | 1.1 |
| | 3 FT | 1.2 | 0.9 | 2.7 | 1.8 | 5.4 | 4.5 | 1.1 | 1.1 |
| HOA, % | T0 | 0.4 | 0.3 | 0.3 | 0.2 | 0.4 | 0.6 | 0.5 | 0.6 |
| | 1 FT | 0.5 | 0.5 | 0.6 | 0.3 | 3.6 | 4.1 | 1.1 | 1.1 |
| | 3 FT | 0.4 | 0.4 | 1.5 | 0.7 | 9.6 | 11.3 | 0.9 | 1.0 |
| Fragments, % | T0 | 0.4 | 0.4 | 0.4 | 0.4 | 0.9 | 0.7 | 0.4 | 0.4 |
| | 1 FT | 0.4 | 0.4 | 0.4 | 0.4 | 0.9 | 0.7 | 0.5 | 0.4 |
| | 3 FT | 0.6 | 0.4 | 0.5 | 0.5 | 0.5 | 0.5 | 0.5 | 0.5 |

1 FT refers to one cycle of Freeze/Thaw; 3 FT refers to three cycles of Freeze/Thaw.

Overall, formulations with glycine and sucrose performed the best for all stress conditions—these formulations showed the highest purity (FIG. 4C) and lower sub-visible and visible particles compared to all other samples, which was especially evident upon stirring stress (FIG. 4D). Compared to the performance of formulations containing PS80, those with PS20 exhibited the lowest performance, particularly during freeze-thaw cycling (FIG. 4A, FIG. 4B, FIG. 4C, and FIG. 4D). Overall, formulations containing glycine, sucrose, and PS80 performed the best under all stress conditions. These formulations demonstrated the highest purity (FIG. 4C) and had fewer sub-visible and visible particles, which was particularly evident under stirring stress (FIG. 4D), compared to all other samples.

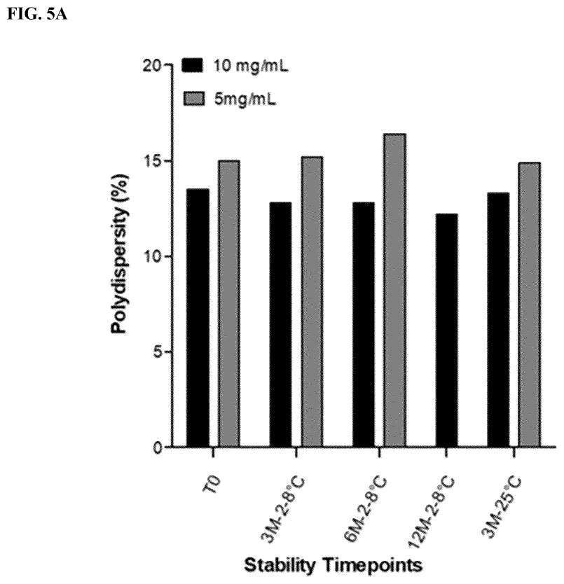

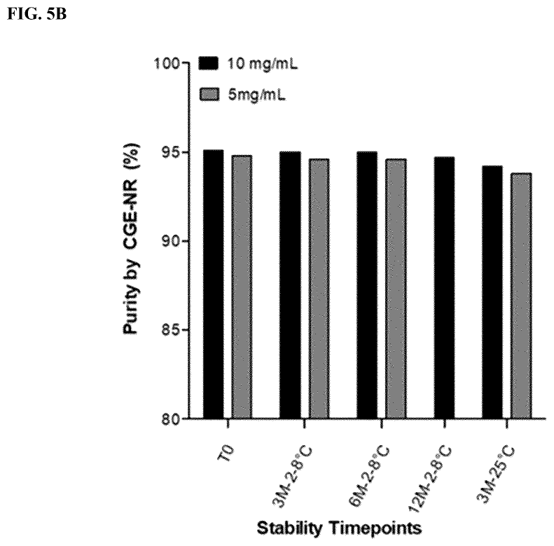

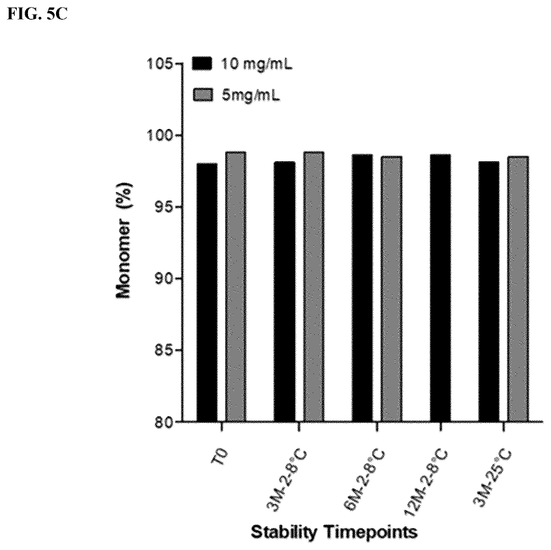

H7-767 at 10 mg/mL in 20 mM acetate, 400 mM glycine, 200 mM sucrose, and 0.05% PS80 at pH 5.0 eliminated visible particle formation along and improved the stability up to 12 months of storage at 2-8° C. as shown by critical quality attributes such as purity by SE-HPLC (monomer %>98%) (FIG. 5C), charge heterogeneity by icIEF (main peak≥59%), size heterogeneity (purity >98% for reduced and >94% for non-reduced; FIG. 5B), relative potency ≥100%, turbidity at ≤6.1, polydispersity at <15% (FIG. 5A), sub-visible particles by MFI within the USP <788> specifications, and maintained the initial protein concentration and pH at 5.0. Additionally, H7-767 at 5 mg/mL in 20 mM acetate, 400 mM glycine, 200 mM sucrose, and 0.05% PS80 at pH 5.0 also performed as a stable formulation at the studied stability conditions although slightly lower-performing compared to the 10 mg/mL protein concentration formulation especially in polydispersity, non-reduced size heterogeneity, and purity (FIG. 5A, FIG. 5B, and FIG. 5C). Hence, glycine as a zwitterion amino acid serves as an osmolyte and stabilizing agent. In combination with sucrose, glycine highly stabilized H7-767 against aggregation.

TABLE 5
Concentration of sub-visible particles by MFI in H7-767 DP formulations

| | | H7-767 formulations, sub-visible particles per mL | | | | | | | |
|---|---|---|---|---|---|---|---|---|---|
| | | | | | | | | 10 | 10 |
| | | | | | | | | mg/mL, | mg/mL, |
| | | 2 | 5 | | | | | 0.05% | 0.05% |
| | | mg/mL, | mg/mL, | | | | | PS80, | PS80, |
| | | 250 mM | 250 mM | 2 | 5 | 2 | 5 | 200 mM | 200 mM |
| Sub- | | sucrose, | sucrose, | mg/mL, | mg/mL, | mg/mL, | mg/mL, | sucrose, | sucrose, |
| visible | | 0.05% | 0.05% | 0.1% | 0.1% | 0.1% | 0.1% | 100 mM | 400 mM |
| particles | Condition | PS80 | PS80 | PS80 | PS80 | PS20 | PS20 | glycine | glycine |
| ≥1 μm | T0 | 75143 | 24740 | 230444 | 302950 | 159067 | 23375 | 7460 | 3125 |
| | 20 min | 117610 | 49251 | 197258 | 100751 | 34748 | 91462 | 14046 | 9418 |
| | stirring | | | | | | | | |
| | 30 min | 373271 | 293462 | 287315 | 171917 | 254166 | 81997 | 25581 | 58677 |
| | stirring | | | | | | | | |
| | 60 min | 373710 | 93183 | 445627 | 189602 | 310153 | 160632 | 78937 | 36362 |
| | stirring | | | | | | | | |
| | 1 FT | 87065 | 11398 | 52463 | 93679 | 43502 | 75575 | 9151 | 9268 |
| | 3 FT | 2114 | 2224 | 6161 | 8133 | 2862 | 5647 | 2485 | 3473 |
| ≥2 μm | T0 | 20913 | 8295 | 77429 | 119300 | 56397 | 9158 | 2774 | 1007 |
| | 20 min | 20737 | 14097 | 50975 | 31413 | 8637 | 31966 | 4157 | 1799 |
| | stirring | | | | | | | | |
| | 30 min | 94725 | 95022 | 76393 | 54532 | 79225 | 30975 | 6652 | 20345 |
| | stirring | | | | | | | | |
| | 60 min | 77333 | 25294 | 114419 | 56789 | 91063 | 55774 | 15102 | 6644 |
| | stirring | | | | | | | | |
| | 1 FT | 23329 | 3368 | 15480 | 35564 | 16829 | 28683 | 2743 | 3180 |
| | 3 FT | 425 | 400 | 2759 | 1362 | 333 | 707 | 523 | 856 |
| ≥5 μm | T0 | 6701 | 2910 | 28356 | 46607 | 20570 | 3676 | 974 | 365 |
| | 20 min | 4598 | 4974 | 15322 | 11667 | 2785 | 12356 | 1391 | 527 |
| | stirring | | | | | | | | |
| | 30 min | 26062 | 31679 | 22743 | 18948 | 26812 | 11687 | 2045 | 8489 |
| | stirring | | | | | | | | |
| | 60 min | 20393 | 8029 | 38330 | 19726 | 25625 | 20919 | 3568 | 1612 |
| | stirring | | | | | | | | |
| | 1 FT | 6838 | 1151 | 4946 | 13648 | 6594 | 11322 | 887 | 1037 |
| | 3 FT | 28 | 26 | 1869 | 142 | 52 | 88 | 32 | 33 |
| ≥10 μm | T0 | 1686 | 760 | 8587 | 13744 | 6231 | 1082 | 260 | 102 |
| | 20 min | 989 | 1582 | 3374 | 3619 | 751 | 3841 | 339 | 141 |
| | stirring | | | | | | | | |
| | 30 min | 5035 | 7519 | 5466 | 5012 | 7120 | 3190 | 508 | 3397 |
| | stirring | | | | | | | | |
| | 60 min | 4407 | 2123 | 10869 | 5497 | 6302 | 5374 | 829 | 60 |
| | stirring | | | | | | | | |
| | 1 FT | 1357 | 288 | 1298 | 4628 | 2369 | 3992 | 238 | 302 |
| | 3 FT | 7 | 11 | 1175 | 31 | 16 | 25 | 10 | 7 |
| ≥25 μm | T0 | 126 | 84 | 832 | 1697 | 673 | 104 | 34 | 12 |
| | 20 min | 62 | 178 | 131 | 380 | 66 | 424 | 28 | 25 |
| | stirring | | | | | | | | |
| | 30 min | 387 | 920 | 397 | 512 | 660 | 325 | 39 | 545 |
| | stirring | | | | | | | | |
| | 60 min | 389 | 283 | 1011 | 565 | 637 | 401 | 70 | 60 |
| | stirring | | | | | | | | |
| | 1 FT | 68 | 19 | 70 | 650 | 306 | 662 | 38 | 44 |
| | 3 FT | 2 | 1 | 1 | 1 | 2 | 1 | 2 | 1 |

### Amino Acid Evaluation

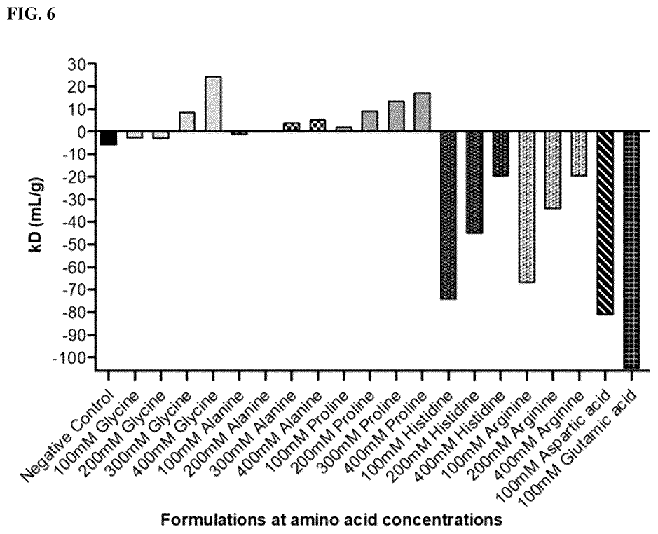

The diffusion interaction coefficient (kD) was determined for formulations containing various concentrations of glycine, alanine, proline, histidine, arginine, aspartic acid, and glutamic acid (see FIG. 6) and 10 mg/mL of H7-767, 20 mM acetate buffer, and 0.05% PS 80, at pH 5. H7-767 in 400 mM glycine formulation exhibited the strongest repulsive protein-protein interaction compared to other studied amino acids at different concentrations (FIG. 6).

### Glycine-Sucrose Optimization Study

For the glycine-sucrose optimization study, a combination of these excipients at different ratios (sucrose at 0 mM to 200 mM and glycine at 0 mM to 500 mM) were evaluated.

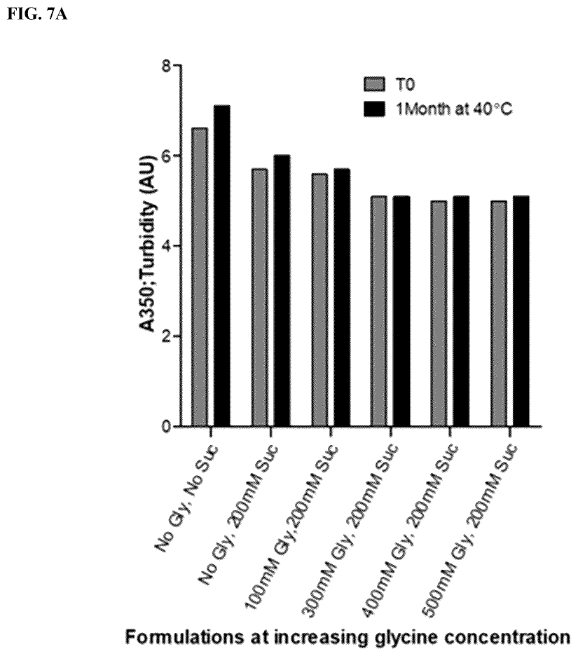

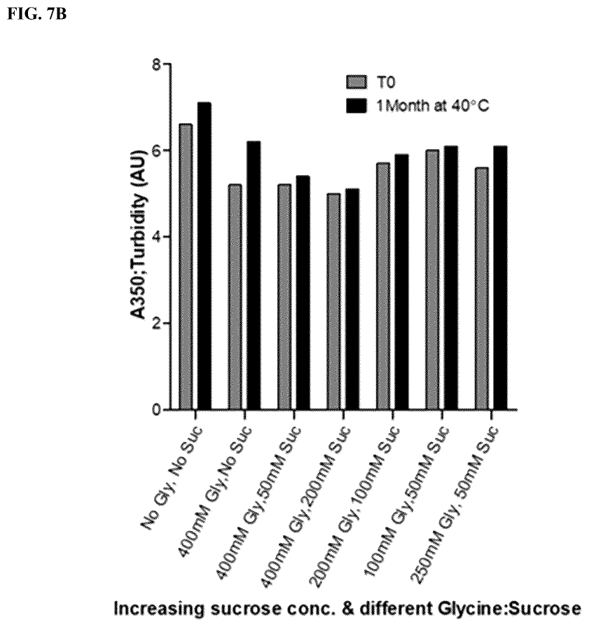

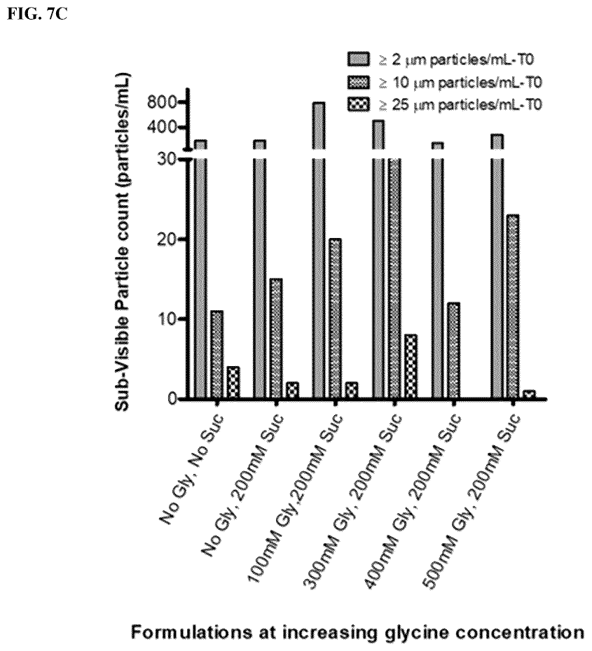

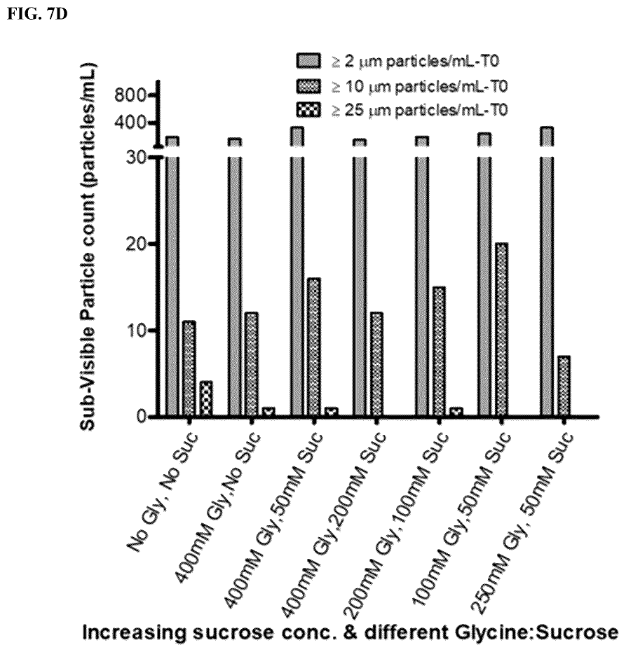

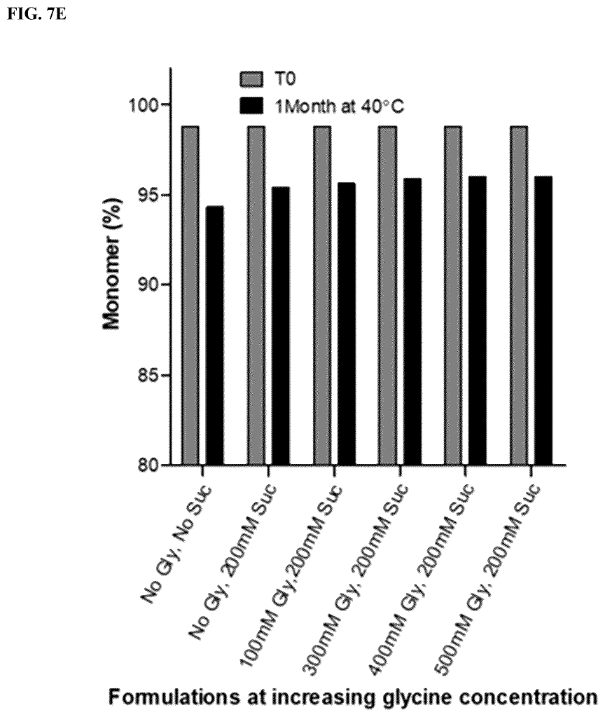

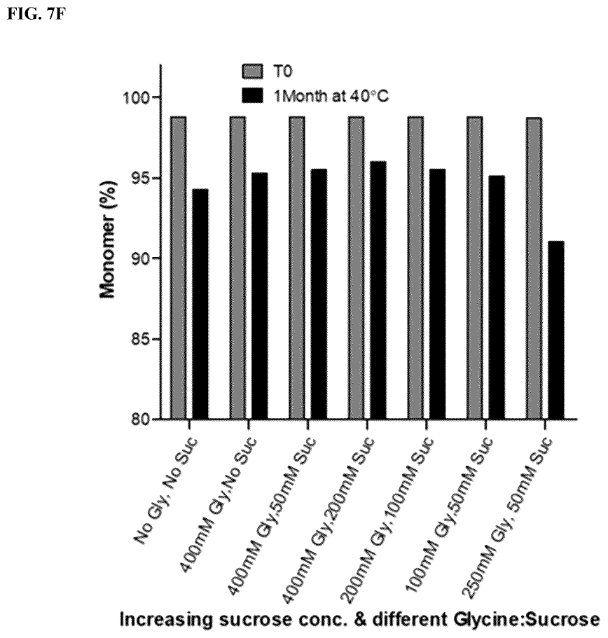

Three formulations comprising 200 mM sucrose and glycine at 300 mM, 400 mM, and 500 mM, respectively, were the most promising formulation candidates for H7-767 due to decreased turbidity (FIG. 7A), reduced sub-visible particles (FIG. 7B), as well as the retention of higher monomer purity (FIG. 7C) even after the stressed condition of storage at 40° C. for two weeks. At the constant glycine concentration of 400 mM without sucrose or with 50 mM sucrose, an increase in turbidity and sub-visible particle count with a slight decrease in purity was observed (FIG. 7B, FIG. 7D, and FIG. 7E, respectfully). Moreover, other molar concentration ratios of glycine to sucrose negatively impacted the critical quality attributes of the molecules as demonstrated by an increase in turbidity, increase in the sub-visible particle count, and decrease in purity of the molecule as shown in FIG. 7B, FIG. 7B, and FIG. 7E. Among these three formulations, the highest thermal unfolding transition temperatures by DSC were observed for 400 mM and 500 mM glycine combination formulations.

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 6
Exemplary Antibodies/Immunoconjugates

| Antibody Name | Heavy chain | Light chain |
|---|---|---|
| 2H7-hIgG4 | 2H7-hIgG4 HC | 2H7-hKappa LC |
| | (SEQ ID NO: 77) | (SEQ ID NO: 78) |
| C51E6- | C51E6-5-hIgG4 HC | C51E6-5-hKappa LC |
| 5-hIgG4 | (SEQ ID NO: 79) | (SEQ ID NO: 80) |
| A2-hIgG4 | A2-hIgG4 HC | A2-hLambda LC |
| | (SEQ ID NO: 81) | (SEQ ID NO: 82) |
| H7-632- | H7-632 HC | H7-632 LC |
| hIgG1-LAGA | (SEQ ID NO: 67) | (SEQ ID NO: 68) |
| H7-767 (also referred | H7-767 HC | H7-632 LC |
| to herein as H7-632- | (SEQ ID NO: 83) | (SEQ ID NO: 68) |
| IgG1-LAGA-IL-2AAEA) | | |

TABLE 7
Sequences

| SEQ | | |
|---|---|---|
| ID | | |
| NO: | Name | Sequence |
| 1 | hIL-2 D20A | APTSSSTKKTQLQLEHLLLALQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 2 | hIL-2 D20N | APTSSSTKKTQLQLEHLLLNLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 3 | hIL-2 R38A | APTSSSTKKTQLQLEHLLLDLQMILNGINNYKNPKLTAMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 4 | hIL-2 R38D | APTSSSTKKTQLQLEHLLLDLQMILNGINNYKNPKLTDMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 5 | hIL-2 R38E | APTSSSTKKTQLQLEHLLLDLQMILNGINNYKNPKLTEMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 6 | hIL-2 R38Q | APTSSSTKKTQLQLEHLLLDLQMILNGINNYKNPKLTQMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 7 | hIL-2 D20I | APTSSSTKKTQLQLEHLLLILQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 8 | hIL-2 D20S | APTSSSTKKTQLQLEHLLLSLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 9 | hIL-2 D20T | APTSSSTKKTQLQLEHLLLTLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 10 | hIL-2 | APTSSSTKKTQLQLEHLLLALQMILNGINNYKNPKLTNMLTFKFYMPKKATELKHLQCLEEELKPLEEV |
| | D20A/R38N | LNLAQSKNFHLRPRDLISNINVIVLELKGSETTFMCEYADETATIVEFLNRWITFCQSIISTLT |
| 11 | hIL-2 | APTSSSTKKTQLQLEHLLLALQMILNGINNYKNPKLTGMLTFKFYMPKKATELKHLQCLEEELKPLEEV |
| | D20A/R38G | LNLAQSKNFHLRPRDLISNINVIVLELKGSETTFMCEYADETATIVEFLNRWITFCQSIISTLT |
| 12 | hIL-2 | APTSSSTKKTQLQLEHLLLALQMILNGINNYKNPKLTHMLTFKFYMPKKATELKHLQCLEEELKPLEEV |
| | D20A/R38H | LNLAQSKNFHLRPRDLISNINVIVLELKGSETTEMCEYADETATIVEFLNRWITFCQSIISTLT |
| 13 | hIL-2 | APTSSSTKKTQLQLEHLLLALQMILNGINNYKNPKLTIMLTFKFYMPKKATELKHLQCLEEELKPLEEV |
| | D20A/R38I | LNLAQSKNFHLRPRDLISNINVIVLELKGSETTFMCEYADETATIVEFLNRWITFCQSIISTLT |
| 14 | hIL-2 | APTSSSTKKTQLQLEHLLLALQMILNGINNYKNPKLTLMLTFKFYMPKKATELKHLQCLEEELKPLEEV |
| | D20A/R38L | LNLAQSKNFHLRPRDLISNINVIVLELKGSETTFMCEYADETATIVEFLNRWITFCQSIISTLT |
| 15 | hIL-2 | APTSSSTKKTQLQLEHLLLALQMILNGINNYKNPKLTMMLTFKFYMPKKATELKHLQCLEEELKPLEEV |
| | D20A/R38M | LNLAQSKNFHLRPRDLISNINVIVLELKGSETTFMCEYADETATIVEFLNRWITFCQSIISTLT |
| 16 | hIL-2 | APTSSSTKKTQLQLEHLLLALQMILNGINNYKNPKLTFMLTFKFYMPKKATELKHLQCLEEELKPLEEV |
| | D20A/R38F | LNLAQSKNFHLRPRDLISNINVIVLELKGSETTEMCEYADETATIVEFLNRWITFCQSIISTLT |
| 17 | hIL-2 | APTSSSTKKTQLQLEHLLLALQMILNGINNYKNPKLTPMLTFKFYMPKKATELKHLQCLEEELKPLEEV |
| | D20A/R38P | LNLAQSKNFHLRPRDLISNINVIVLELKGSETTFMCEYADETATIVEFLNRWITFCQSIISTLT |
| 18 | hIL-2 | APTSSSTKKTQLQLEHLLLALQMILNGINNYKNPKLTSMLTFKFYMPKKATELKHLQCLEEELKPLEEV |
| | D20A/R38S | LNLAQSKNFHLRPRDLISNINVIVLELKGSETTEMCEYADETATIVEFLNRWITFCQSIISTLT |
| 19 | hIL-2 | APTSSSTKKTQLQLEHLLLALQMILNGINNYKNPKLTTMLTFKFYMPKKATELKHLQCLEEELKPLEEV |
| | D20A/R38T | LNLAQSKNFHLRPRDLISNINVIVLELKGSETTFMCEYADETATIVEFLNRWITFCQSIISTLT |
| 20 | hIL-2 | APTSSSTKKTQLQLEHLLLALQMILNGINNYKNPKLTWMLTFKFYMPKKATELKHLQCLEEELKPLEEV |
| | D20A/R38W | LNLAQSKNFHLRPRDLISNINVIVLELKGSETTFMCEYADETATIVEFLNRWITFCQSIISTLT |
| 21 | hIL-2 | APTSSSTKKTQLQLEHLLLALQMILNGINNYKNPKLTYMLTFKFYMPKKATELKHLQCLEEELKPLEEV |
| | D20A/R38Y | LNLAQSKNFHLRPRDLISNINVIVLELKGSETTFMCEYADETATIVEFLNRWITFCQSIISTLT |
| 22 | hIL-2 | APTSSSTKKTQLQLEHLLLALQMILNGINNYKNPKLTVMLTFKFYMPKKATELKHLQCLEEELKPLEEV |
| | D20A/R38V | LNLAQSKNFHLRPRDLISNINVIVLELKGSETTFMCEYADETATIVEFLNRWITFCQSIISTLT |
| 23 | hIL-2 | APTSSSTKKTQLQLEHLLLALQMILNGINNYKNPKLTAMLTFKFYMPKKATELKHLQCLEEELKPLEEV |
| | D20A/R38A | LNLAQSKNFHLRPRDLISNINVIVLELKGSETTFMCEYADETATIVEFLNRWITFCQSIISTLT |
| 24 | hIL-2 | APTSSSTKKTQLQLEHLLLALQMILNGINNYKNPKLTQMLTFKFYMPKKATELKHLQCLEEELKPLEEV |
| | D20A/R38Q | LNLAQSKNFHLRPRDLISNINVIVLELKGSETTEMCEYADETATIVEFLNRWITFCQSIISTLT |
| 25 | hIL-2 | APTSSSTKKTQLQLEHLLLALQMILNGINNYKNPKLTEMLTFKFYMPKKATELKHLQCLEEELKPLEEV |
| | D20A/R38E | LNLAQSKNFHLRPRDLISNINVIVLELKGSETTFMCEYADETATIVEFLNRWITFCQSIISTLT |
| 26 | hIL-2 | APTSSSTKKTQLQLEHLLLALQMILNGINNYKNPKLTDMLTFKFYMPKKATELKHLQCLEEELKPLEEV |
| | D20A/R38D | LNLAQSKNFHLRPRDLISNINVIVLELKGSETTFMCEYADETATIVEFLNRWITFCQSIISTLT |
| 27 | hIL-2 | APTSSSTKKTQLQLEHLLLNLQMILNGINNYKNPKLTEMLTFKFYMPKKATELKHLQCLEEELKPLEEV |
| | D20N/R38E | LNLAQSKNFHLRPRDLISNINVIVLELKGSETTFMCEYADETATIVEFLNRWITFCQSIISTLT |
| 28 | hIL-2 | APTSSSTKKTQLQLEHLLLQLQMILNGINNYKNPKLTEMLTFKFYMPKKATELKHLQCLEEELKPLEEV |
| | D20Q/R38E | LNLAQSKNFHLRPRDLISNINVIVLELKGSETTFMCEYADETATIVEFLNRWITFCQSIISTLT |
| 29 | hIL-2 | APTSSSTKKTQLQLEHLLLELQMILNGINNYKNPKLTEMLTFKFYMPKKATELKHLQCLEEELKPLEEV |
| | D20E/R38E | LNLAQSKNFHLRPRDLISNINVIVLELKGSETTFMCEYADETATIVEFLNRWITFCQSIISTLT |
| 30 | hIL-2 | APTSSSTKKTQLQLEHLLLGLQMILNGINNYKNPKLTEMLTFKFYMPKKATELKHLQCLEEELKPLEEV |
| | D20G/R38E | LNLAQSKNFHLRPRDLISNINVIVLELKGSETTFMCEYADETATIVEFLNRWITFCQSIISTLT |
| 31 | hIL-2 | APTSSSTKKTQLQLEHLLLILQMILNGINNYKNPKLTEMLTFKFYMPKKATELKHLQCLEEELKPLEEV |
| | D20I/R38E | LNLAQSKNFHLRPRDLISNINVIVLELKGSETTEMCEYADETATIVEFLNRWITFCQSIISTLT |
| 32 | hIL-2 | APTSSSTKKTQLQLEHLLLMLQMILNGINNYKNPKLTEMLTFKFYMPKKATELKHLQCLEEELKPLEEV |
| | D20M/R38E | LNLAQSKNFHLRPRDLISNINVIVLELKGSETTFMCEYADETATIVEFLNRWITFCQSIISTLT |
| 33 | hIL-2 | APTSSSTKKTQLQLEHLLLTLQMILNGINNYKNPKLTEMLTFKFYMPKKATELKHLQCLEEELKPLEEV |
| | D20T/R38E | LNLAQSKNFHLRPRDLISNINVIVLELKGSETTEMCEYADETATIVEFLNRWITFCQSIISTLT |
| 34 | hIL-2 | APTSSSTKKTQLQLEHLLLVLQMILNGINNYKNPKLTEMLTFKFYMPKKATELKHLQCLEEELKPLEEV |
| | D20V/R38E | LNLAQSKNFHLRPRDLISNINVIVLELKGSETTFMCEYADETATIVEFLNRWITFCQSIISTLT |
| 35 | hIL-2 | APTSSSTKKTQLQLEHLLLALQMILNGINNYKNPKLTEMLTFKFYMPKKATELKHLQCLEEELKPLEEV |
| | D20A/R38E/C125A | LNLAQSKNFHLRPRDLISNINVIVLELKGSETTFMCEYADETATIVEFLNRWITFAQSIISTLT |
| 36 | hIL-2 | APASSSTKKTQLQLEHLLLALQMILNGINNYKNPKLTEMLTFKFYMPKKATELKHLQCLEEELKPLEEV |
| | T3A/D20A/R38E | LNLAQSKNFHLRPRDLISNINVIVLELKGSETTFMCEYADETATIVEFLNRWITFCQSIISTLT |
| 37 | hIL-2 | APASSSTKKTQLQLEHLLLALQMILNGINNYKNPKLTEMLTFKFYMPKKATELKHLQCLEEELKPLEEV |
| | T3A/D20A/R38E/ | LNLAQSKNFHLRPRDLISNINVIVLELKGSETTFMCEYADETATIVEFLNRWITFAQSIISTLT |
| | C125A | |
| | (IL-2-AAEA) | |
| 38 | hIL-2 Δ1- | SSSTKKTQLQLEHLLLALQMILNGINNYKNPKLTEMLTFKFYMPKKATELKHLQCLEEELKPLEEVLNL |
| | 3APT/D20A/R38E | AQSKNFHLRPRDLISNINVIVLELKGSETTFMCEYADETATIVEFLNRWITFCQSIISTLT |
| 39 | hIL-2 Δ1- | SSSTKKTQLQLEHLLLALQMILNGINNYKNPKLTEMLTFKFYMPKKATELKHLQCLEEELKPLEEVLNL |
| | 3APT/D20A/R38E/ | AQSKNFHLRPRDLISNINVIVLELKGSETTEMCEYADETATIVEFLNRWITFAQSIISTLT |
| | C125A | |
| 40 | hIL-2 | APTSSSTKKTQLQLEHLLLSLQMILNGINNYKNPKLTEMLTFKFYMPKKATELKHLQCLEEELKPLEEV |
| | R38E/D20S | LNLAQSKNFHLRPRDLISNINVIVLELKGSETTFMCEYADETATIVEFLNRWITFCQSIISTLT |
| 41 | hIL-2 | APTSSSTKKTQLQLEHLLLALQMILNGINNYKNPKLTKMLTFKFYMPKKATELKHLQCLEEELKPLEEV |
| | R38K/D20A | LNLAQSKNFHLRPRDLISNINVIVLELKGSETTFMCEYADETATIVEFLNRWITFCQSIISTLT |
| 42 | WT hIL-2 | APTSSSTKKTQLQLEHLLLDLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 43 | 2H7 VH | EVQLLESGGGLVQPGGSLRLSCAASGFTFKDYCMTWVRQAPGKGLEWVSAIVYSGGSTYYADSVKGRET |
| | | ISRDNSKNTLYLQMNNLRAEDTAVYYCAKYTRASYFYDAMDVWGQGTTVTVSS |
| 44 | 2H7 VL | EIVLTQSPGTLSLSPGERATLSCRASQSIGKSFLAWYQQKPGQAPRLLIYDASTRAADIPARESGSGSG |
| | | TDFTLTISSLEPEDFAVYYCQQYYDWPPLSFGGGTKVEIK |
| 45 | 2H7 HCDR1 | GFTFKDYCMT |
| 46 | 2H7 HCDR2 | AIVYSGGSTYYADSVKG |
| 47 | 2H7 HCDR3 | YTRASYFYDAMDV |
| 48 | 2H7 LCDR1 | RASQSIGKSFLA |
| 49 | 2H7 LCDR2 | DASTRAA |
| 50 | 2H7 LCDR3 | QQYYDWPPLS |
| 51 | C51E6-5 VH | QVQLVQSGSELKKPGASVKVSCKASGYSLYGTSMHWVRQAPGOGLEWMGYISPFTGRATYAQGFTGRFV |
| | | FSLDTSVSTAYLQISSLKAEDTAVYYCARDYDYRYYYAMDYWGQGTTVTVSS |
| 52 | C51E6-5 VL | EIVLTQSPDFQSVTPKEKVTITCTASESVPPQFLHWYQQKPDQSPKLLIYASRERASGVPSRESGSGSG |
| | | TDFTLTINSLEAEDAATYYCHQFHRSPLTFGGGTKLEIK |
| 53 | C51E6-5 HCDR1 | GYSLYGTSMH |
| 54 | C51E6-5 HCDR2 | YISPFTGRATYAQGFTG |
| 55 | C51E6-5 HCDR3 | DYDYRYYYAMDY |
| 56 | C51E6-5 LCDR1 | TASESVPPQFLH |
| 57 | C51E6-5 LCDR2 | ASRERAS |
| 58 | C51E6-5 LCDR3 | HQFHRSPLT |
| 59 | A2 VH | DVQLVESGGGLVQPGGSLRLSCAASGFTFDISAMSWVRQAPGKGLEWVSTISGSAYSTYYADSVKGRET |
| | | ISRDNSKSTLYLQMNSLRAEDTAVYYCAREIFSDYWGLGTLVTVSS |
| 60 | A2 VL | QSVLTQPPSASGTPGQRVTISCSGSTSNIGRESVYWYQQLPGTAPKLLIYSNVQRPSGAPNRESGSKSG |
| | (OMC479p1. | TSASLAISGLOSEDEADYYCGTWDDSLNGWVFGGGTKLTVL |
| | A2VL) | |
| 61 | A2 HCDR1 | GFTEDISAMS |
| 62 | A2 HCDR2 | TISGSAYSTYYADSVKG |
| 63 | A2 HCDR3 | EIFSDY |
| 64 | A2 LCDR1 | SGSTSNIGRESVY |
| 65 | A2 LCDR2 | SNVQRPS |
| 66 | A2 LCDR3 | GTWDDSLNGWV |
| 67 | H7-632 HC | EVQLLESGGGLVQPGGSLRLSCAASGFTFKSYAMHWVRQAPGKGLEWVSAIVYSGGSTYYADSVKGRET |
| | CDRs solid | 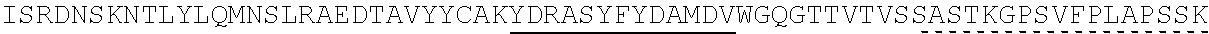 |
| | underlined | 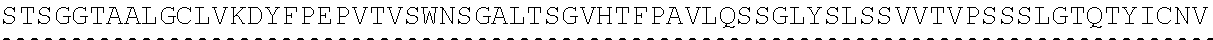 |
| | Constant | 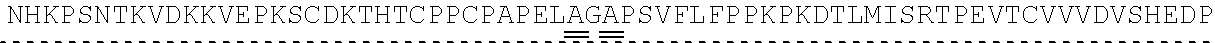 |
| | region dashed | 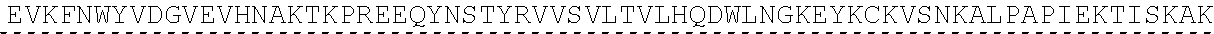 |
| | underlined | 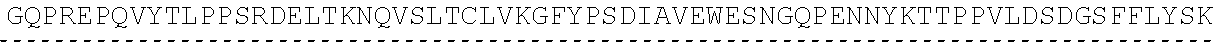 |
| | | 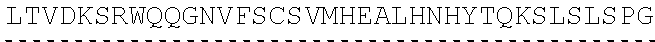 |
| 68 | H7-632 LC | EIVLTQSPGTLSLSPGERATLSCRASQSISSSFLAWYQQKPGQAPRLLIYDASDRATGIPDRESGSGSG |
| | CDRs solid | 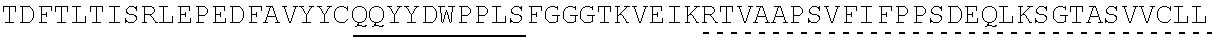 |
| | underlined | 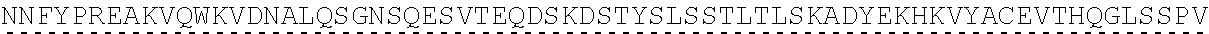 |
| | Constant | 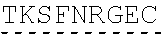 |
| | region dashed | |
| | underlined | |
| 69 | H7-632 VH | EVQLLESGGGLVQPGGSLRLSCAASGFTFKSYAMHWVRQAPGKGLEWVSAIVYSGGSTYYADSVKGRFT |
| | | ISRDNSKNTLYLQMNSLRAEDTAVYYCAKYDRASYFYDAMDVWGQGTTVTVSS |
| 70 | H7-632 VL | EIVLTQSPGTLSLSPGERATLSCRASQSISSSFLAWYQQKPGQAPRLLIYDASDRATGIPDRESGSGSG |
| | | TDFTLTISRLEPEDFAVYYCQQYYDWPPLSFGGGTKVEIK |
| 71 | H7-632 HCDR1 | GFTFKSYAMH |
| 72 | H7-632 HCDR2 | AIVYSGGSTYYADSVKG |
| 73 | H7-632 HCDR3 | YDRASYFYDAMDV |
| 74 | H7-632 LCDR1 | RASQSISSSFLA |
| 75 | H7-632 LCDR2 | DASDRAT |
| 76 | H7-632 LCDR3 | QQYYDWPPLS |
| 77 | 2H7-hIgG4 HC | EVQLLESGGGLVQPGGSLRLSCAASGFTFKDYCMTWVRQAPGKGLEWVSAIVYSGGSTYYADSVKGRFT |
| | | ISRDNSKNTLYLQMNNLRAEDTAVYYCAKYTRASYFYDAMDVWGQGTTVTVSSASTKGPSVFPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFLGGPSVFLFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 78 | 2H7-hkappa LC | EIVLTQSPGTLSLSPGERATLSCRASQSIGKSFLAWYQQKPGQAPRLLIYDASTRAADIPARFSGSGSG |
| | | TDFTLTISSLEPEDFAVYYCQQYYDWPPLSFGGGTKVEIKRTVAAPSVFIFPPSDEQLKSGTASVVCLL |
| | | NNFYPREAKVQWKVDNALQSGNSQESVTEQDSKDSTYSLSSTLTLSKADYEKHKVYACEVTHQGLSSPV |
| | | TKSENRGEC |
| 79 | C51E6-5-hIgG4 | QVQLVQSGSELKKPGASVKVSCKASGYSLYGTSMHWVRQAPGOGLEWMGYISPFTGRATYAQGFTGRFV |
| | HC | FSLDTSVSTAYLQISSLKAEDTAVYYCARDYDYRYYYAMDYWGQGTTVTVSSASTKGPSVEPLAPCSRS |
| | | TSESTAALGCLVKDYFPEPVTVSWNSGALTSGVHTFPAVLOSSGLYSLSSVVTVPSSSLGTKTYTCNVD |
| | | HKPSNTKVDKRVESKYGPPCPPCPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDGSFFLYSRLTVD |
| | | KSRWQEGNVESCSVMHEALHNHYTQKSLSLSLG |
| 80 | C51E6-5-hKappa | EIVLTQSPDFQSVTPKEKVTITCTASESVPPQFLHWYQQKPDQSPKLLIYASRERASGVPSRESGSGSG |
| | LC | TDFTLTINSLEAEDAATYYCHQFHRSPLTFGGGTKLEIKRTVAAPSVFIFPPSDEQLKSGTASVVCLLN |
| | | NFYPREAKVQWKVDNALQSGNSQESVTEQDSKDSTYSLSSTLTLSKADYEKHKVYACEVTHQGLSSPVT |
| | | KSENRGEC |
| 81 | A2-hIgG4 HC | DVQLVESGGGLVQPGGSLRLSCAASGFTFDISAMSWVRQAPGKGLEWVSTISGSAYSTYYADSVKGRFT |
| | | ISRDNSKSTLYLQMNSLRAEDTAVYYCAREIFSDYWGLGTLVTVSSASTKGPSVFPLAPCSRSTSESTA |
| | | ALGCLVKDYFPEPVTVSWNSGALTSGVHTFPAVLOSSGLYSLSSVVTVPSSSLGTKTYTCNVDHKPSNT |
| | | KVDKRVESKYGPPCPPCPAPEFLGGPSVFLFPPKPKDTLMISRTPEVTCVVVDVSQEDPEVQENWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 82 | A2-hLambda LC | QSVLTQPPSASGTPGQRVTISCSGSTSNIGRESVYWYQQLPGTAPKLLIYSNVQRPSGAPNRFSGSKSG |
| | | TSASLAISGLQSEDEADYYCGTWDDSLNGWVFGGGTKLTVLGQPKAAPSVTLFPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 83 | H7-767 HC | EVQLLESGGGLVQPGGSLRLSCAASGFTFKSYAMHWVRQAPGKGLEWVSAIVYSGGSTYYADSVKGRFT |
| | | ISRDNSKNTLYLQMNSLRAEDTAVYYCAKYDRASYFYDAMDVWGQGTTVTVSSASTKGPSVFPLAPSSK |
| | | STSGGTAALGCLVKDYFPEPVTVSWNSGALTSGVHTFPAVLQSSGLYSLSSVVTVPSSSLGTQTYICNV |
| | | NHKPSNTKVDKKVEPKSCDKTHTCPPCPAPELAGAPSVELFPPKPKDTLMISRTPEVTCVVVDVSHEDP |
| | | EVKFNWYVDGVEVHNAKTKPREEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLALQMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 84 | 2H7 VH | GAGGTGCAGCTGCTGGAAAGCGGCGGCGGACTGGTGCAGCCTGGAGGCAGCCTGCGGCTGTCTTGTGCC |
| | | GCTTCTGGCTTCACCTTCAAGGACTACTGCATGACCTGGGTCAGACAGGCCCCTGGCAAGGGCCTCGAG |
| | | TGGGTGTCCGCCATCGTGTACAGCGGCGGGTCAACATACTACGCCGACAGCGTGAAGGGCAGATTCACA |
| | | ATCAGCAGAGATAACAGCAAGAACACCCTGTACCTGCAGATGAACAACCTGAGAGCTGAAGATACCGCC |
| | | GTGTACTACTGCGCCAAGTACACCAGAGCCAGCTACTTCTACGACGCCATGGACGTGTGGGGCCAGGGC |
| | | ACCACCGTGACAGTGTCCTCAT |
| 85 | 2H7 VL | GAGATCGTGCTGACCCAGTCTCCTGGCACCCTGAGCCTGAGCCCTGGCGAGAGAGCTACACTGTCATGC |
| | | AGAGCCTCTCAGAGCATCGGCAAGAGCTTCCTGGCCTGGTACCAGCAAAAGCCTGGACAGGCCCCTAGA |
| | | CTGCTGATCTACGACGCCAGCACCAGAGCCGCTGATATCCCCGCCAGATTCAGCGGATCTGGCAGCGGC |
| | | ACTGATTTCACCCTCACCATCAGCAGCCTGGAACCCGAGGACTTCGCCGTGTACTACTGCCAGCAGTAC |
| | | TACGACTGGCCCCCCCTGTCTTTTGGCGGAGGCACAAAGGTGGAAATCAAG |
| 86 | C51E6-5 VH | CAGGTTCAGCTGGTTCAGTCTGGCAGCGAGCTGAAGAAACCTGGCGCCTCTGTGAAGGTGTCCTGCAAG |
| | | GCCTCTGGCTACAGCCTGTACGGCACCTCTATGCACTGGGTCCGACAGGCTCCAGGACAGGGACTTGAG |
| | | TGGATGGGCTACATCAGCCCCTTTACCGGCAGAGCCACATACGCCCAGGGCTTCACAGGCAGATTCGTG |
| | | TTCAGCCTGGACACCAGCGTGTCCACAGCCTACCTGCAGATCAGCTCTCTGAAGGCCGAGGACACCGCC |
| | | GTGTACTACTGCGCCAGAGACTACGACTACCGGTACTACTATGCCATGGACTACTGGGGCCAGGGCACC |
| | | ACAGTTACAGTGTCCTCA |
| 87 | C51E6-5 VL | GAAATTGTGCTGACACAGAGCCCCGACTTCCAGAGCGTGACCCCTAAAGAAAAAGTGACCATCACCTGT |
| | | ACCGCCAGCGAGTCCGTGCCTCCTCAGTTCCTGCATTGGTATCAGCAGAAGCCCGATCAGAGCCCCAAG |
| | | CTGCTGATCTACGCCAGCAGAGAAAGAGCCAGCGGCGTCCCAAGCAGATTTTCTGGCTCTGGCAGCGGC |
| | | ACCGACTTCACCCTGACAATCAATAGCCTGGAAGCCGAGGACGCCGCCACCTACTACTGCCACCAGTTT |
| | | CACAGAAGCCCTCTGACCTTTGGCGGAGGCACCAAGCTGGAAATCAAG |
| 88 | A2 VH | GACGTGCAGCTGGTGGAAAGCGGCGGAGGCCTGGTCCAGCCCGGCGGCTCTCTGAGACTGAGCTGCGCC |
| | | GCCAGCGGCTTCACCTTCGACATCAGCGCCATGAGCTGGGTGCGGCAGGCCCCTGGCAAGGGCCTGGAA |
| | | TGGGTCAGCACAATCAGCGGATCTGCCTACAGCACCTACTACGCCGACAGCGTGAAGGGCAGATTCACC |
| | | ATCTCAAGAGATAACAGCAAGAGCACCCTGTACCTGCAGATGAACAGCCTGCGGGCCGAGGACACCGCC |
| | | GTGTACTACTGCGCCAGAGAGATCTTCAGCGACTACTGGGGCTTGGGCACCCTGGTGACAGTGTCCTCA |
| 89 | A2 VL | CAAAGCGTGCTGACACAGCCCCCCAGCGCTTCTGGCACCCCTGGCCAGAGAGTGACCATCTCATGCAGC |
| | | GGGTCAACAAGCAACATCGGCAGAGAGAGCGTGTACTGGTACCAGCAGCTGCCTGGAACCGCCCCTAAG |
| | | CTGCTGATCTACAGCAACGTGCAGCGGCCTAGCGGCGCCCCTAACAGATTCAGCGGCAGCAAGAGCGGC |
| | | ACCAGCGCCAGCCTGGCCATCAGCGGCCTGCAGAGCGAGGACGAGGCCGACTACTACTGCGGCACATGG |
| | | GACGACAGCCTGAACGGCTGGGTGTTCGGCGGCGGAACTAAGCTGACCGTCCTA |
| 90 | 2H7-hIgG4-df- | EVQLLESGGGLVQPGGSLRLSCAASGFTFKDYCMTWVRQAPGKGLEWVSAIVYSGGSTYYADSVKGRET |
| | hIL-2 | ISRDNSKNTLYLQMNNLRAEDTAVYYCAKYTRASYFYDAMDVWGQGTTVTVSSASTKGPSVFPLAPCSR |
| | (D20A/R38E) HC | STSESTAALGCLVKDYFPEPVTVSWNSGALTSGVHTFPAVLQSSGLYSLSSVVTVPSSSLGTKTYTCNV |
| | hIL-2 in | DHKPSNTKVDKRVESKYGPPCPPCPAPEFLGGPSVELFPPKPKDTLMISRTPEVTCVVVDVSQEDPEVQ |
| | italics | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPTSSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFCQSIISTLT |
| 91 | A2-hIgG4-df- | DVQLVESGGGLVQPGGSLRLSCAASGFTFDISAMSWVRQAPGKGLEWVSTISGSAYSTYYADSVKGRET |
| | hIL-2 | ISRDNSKSTLYLQMNSLRAEDTAVYYCAREIFSDYWGLGTLVTVSSASTKGPSVFPLAPCSRSTSESTA |
| | (D20A/R38E) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFCQSIISTLT |
| 92 | 2H7-hIgG4-df- | EVQLLESGGGLVQPGGSLRLSCAASGFTFKDYCMTWVRQAPGKGLEWVSAIVYSGGSTYYADSVKGRFT |
| | hIL-2 | ISRDNSKNTLYLQMNNLRAEDTAVYYCAKYTRASYFYDAMDVWGQGTTVTVSSASTKGPSVEPLAPCSR |
| | (T3A/D20A/R38E/ | STSESTAALGCLVKDYFPEPVTVSWNSGALTSGVHTFPAVLQSSGLYSLSSVVTVPSSSLGTKTYTCNV |
| | C125A) HC | DHKPSNTKVDKRVESKYGPPCPPCPAPEFLGGPSVELFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFAQSIISTLT |
| 93 | 2H7-hIgG1- | EVQLLESGGGLVQPGGSLRLSCAASGFTFKDYCMTWVRQAPGKGLEWVSAIVYSGGSTYYADSVKGRET |
| | LAGA-df-hIL-2 | ISRDNSKNTLYLQMNNLRAEDTAVYYCAKYTRASYFYDAMDVWGQGTTVTVSSASTKGPSVFPLAPSSK |
| | (T3A/D20A/R38E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLALQMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 94 | 2H7-hIgG1- | EVQLLESGGGLVQPGGSLRLSCAASGFTFKDYCMTWVRQAPGKGLEWVSAIVYSGGSTYYADSVKGRFT |
| | LAGA--df-hIL-2 | ISRDNSKNTLYLQMNNLRAEDTAVYYCAKYTRASYFYDAMDVWGQGTTVTVSSASTKGPSVFPLAPCSR |
| | (T3A/D20A/R38E/ | STSESTAALGCLVKDYFPEPVTVSWNSGALTSGVHTFPAVLOSSGLYSLSSVVTVPSSSLGTKTYTCNV |
| | C125A) HC | DHKPSNTKVDKRVESKYGPPCPPCPAPEFAGAPSVELFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFAQSIISTLT |
| 95 | A2-hIgG4-df- | DVQLVESGGGLVQPGGSLRLSCAASGFTFDISAMSWVRQAPGKGLEWVSTISGSAYSTYYADSVKGRFT |
| | hIL-2 | ISRDNSKSTLYLQMNSLRAEDTAVYYCAREIFSDYWGLGTLVTVSSASTKGPSVFPLAPCSRSTSESTA |
| | (D20S/R38E) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVELFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLSLOMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFCQSIISTLT |
| 96 | C51E6-5-hIgG4- | QVQLVQSGSELKKPGASVKVSCKASGYSLYGTSMHWVRQAPGOGLEWMGYISPFTGRATYAQGFTGREV |
| | df-hIL-2 | FSLDTSVSTAYLQISSLKAEDTAVYYCARDYDYRYYYAMDYWGQGTTVTVSSASTKGPSVEPLAPCSRS |
| | (D20A/R38E) HC | TSESTAALGCLVKDYFPEPVTVSWNSGALTSGVHTFPAVLQSSGLYSLSSVVTVPSSSLGTKTYTCNVD |
| | hIL-2 in | HKPSNTKVDKRVESKYGPPCPPCPAPEFLGGPSVFLFPPKPKDTLMISRTPEVTCVVVDVSQEDPEVOF |
| | italics | NWYVDGVEVHNAKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLGKAPTSSSTKKTQLQLEHLLLALQMILNGINNYKNPK |
| | | LTEMLTFKFYMPKKATELKHLQCLEEELKPLEEVLNLAQSKNFHLRPRDLISNINVIVLELKGSETTEM |
| | | CEYADETATIVEFLNRWITFCQSIISTLT |
| 97 | C51E6-5-hIgG4- | QVQLVQSGSELKKPGASVKVSCKASGYSLYGTSMHWVRQAPGOGLEWMGYISPFTGRATYAQGETGREV |
| | LAGA-df-hIL-2 | FSLDTSVSTAYLQISSLKAEDTAVYYCARDYDYRYYYAMDYWGQGTTVTVSSASTKGPSVFPLAPCSRS |
| | (T3A/D20A/R38E/ | TSESTAALGCLVKDYFPEPVTVSWNSGALTSGVHTFPAVLQSSGLYSLSSVVTVPSSSLGTKTYTCNVD |
| | C125A) HC | HKPSNTKVDKRVESKYGPPCPPCPAPEFAGAPSVELFPPKPKDTLMISRTPEVTCVVVDVSQEDPEVQF |
| | hIL-2 in | NWYVDGVEVHNAKTKPREEQFNSTYRVVSVLTVLHQDWLNGKEYKCKVSNKGLPSSIEKTISKAKGQPR |
| | italics | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLGKAPASSSTKKTQLQLEHLLLALQMILNGINNYKNPK |
| | | LTEMLTFKFYMPKKATELKHLQCLEEELKPLEEVLNLAQSKNFHLRPRDLISNINVIVLELKGSETTEM |
| | | CEYADETATIVEFLNRWITFAQSIISTLT |
| 98 | 2H7-hIgG1-df- | EVQLLESGGGLVQPGGSLRLSCAASGFTFKDYCMTWVRQAPGKGLEWVSAIVYSGGSTYYADSVKGRET |
| | hIL-2 | ISRDNSKNTLYLQMNNLRAEDTAVYYCAKYTRASYFYDAMDVWGQGTTVTVSSASTKGPSVFPLAPSSK |
| | (T3A/D20A/R38E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELLGGPSVFLFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLALQMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 99 | 2H7-hIgG1- | EVQLLESGGGLVQPGGSLRLSCAASGFTFKDYCMTWVRQAPGKGLEWVSAIVYSGGSTYYADSVKGRFT |
| | LAGA-df-hIL2 | ISRDNSKNTLYLQMNNLRAEDTAVYYCAKYTRASYFYDAMDVWGQGTTVTVSSASTKGPSVFPLAPSSK |
| | (T3A/D20S/R38E/ | STSGGTAALGCLVKDYFPEPVTVSWNSGALTSGVHTFPAVLOSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLSLOMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |

What is claimed:

1. A stable pharmaceutical formulation comprising:
18 mM to 22 mM of acetate;
50 mM to 250 mM of sucrose;
100 mM to 500 mM of glycine;
0.05% to 0.1% of PS80; and
5 mg/mL to 10 mg/mL of an anti-human PD-1 (hPD-1) antibody-modified human interleukin-2 (hIL-2) immunoconjugate comprising:
a modified hIL-2 protein comprising the amino acid sequence of SEQ ID NO: 25; and
an anti-hPD-1 antibody, or antigen-binding fragment thereof, that immunospecifically binds to hPD-1, wherein the antibody or antigen-binding fragment thereof, comprises a heavy chain complementarity determining region 1 (CDR1) comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.

2. The stable pharmaceutical formulation of claim 1, comprising:
20 mM of acetate;
200 mM of sucrose;
400 mM of glycine; and
0.05% of PS80.

3. The stable pharmaceutical formulation of claim 1, comprising 10 mg/mL of the anti-hPD-1 antibody-modified hIL-2 immunoconjugate.

4. The stable pharmaceutical formulation of claim 1, wherein the pH is 4.5 to 5.5.

5. The stable pharmaceutical formulation of claim 4, wherein the pH is 5.0.

6. The stable pharmaceutical formulation of claim 1, wherein the modified hIL-2 protein further comprises a deletion or substitution at amino acid position 3.

7. The stable pharmaceutical formulation of claim 6, wherein the substitution at amino acid position 3 of the modified hIL-2 protein is T3A.

8. The stable pharmaceutical formulation of claim 7, wherein the modified hIL-2 protein comprises the amino acid sequence of SEQ ID NO: 36.

9. The stable pharmaceutical formulation of claim 1, wherein the modified hIL-2 protein further comprises a deletion or substitution at amino acid position 125.

10. The stable pharmaceutical formulation of claim 9, wherein the substitution at amino acid position 125 is C125A.

11. The stable pharmaceutical formulation of claim 10, wherein the modified hIL-2 protein comprises the amino acid sequence of SEQ ID NO: 37.

12. The stable pharmaceutical formulation of claim 1, wherein the anti-hPD-1 antibody, or antigen-binding fragment thereof, comprises a heavy chain variable region (VH) comprising the amino acid sequence of SEQ ID NO: 69 and a light chain variable region (VL) comprising the amino acid sequence of SEQ ID NO: 70.

13. The stable pharmaceutical formulation of claim 1, wherein the anti-hPD-1 antibody, or antigen-binding fragment thereof, comprises an IgG1 heavy chain constant region.

14. The stable pharmaceutical formulation of claim 13, wherein the anti-hPD-1 antibody, or antigen-binding fragment thereof, comprises an L235A substitution and a G237A substitution, according to EU numbering.

15. The stable pharmaceutical formulation of claim 1, wherein the anti-hPD-1 antibody, or antigen-binding fragment thereof, comprises a heavy chain (HC) comprising the amino acid sequence of SEQ ID NO: 67 and a light chain (LC) comprising the amino acid sequence of SEQ ID NO: 68.

16. The stable pharmaceutical formulation of claim 1, comprising:
18 mM to 22 mM of acetate;
50 mM to 250 mM of sucrose;
100 mM to 500 mM of glycine;
0.05% to 0.1% of PS80; and
5 mg/mL to 10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a HC comprising the amino acid sequence of SEQ ID NO: 67 and a LC comprising the amino acid sequence of SEQ ID NO: 68.

17. The stable pharmaceutical formulation of claim 1, wherein the anti-hPD-1 antibody-modified hIL-2 immunoconjugate comprises:
a light chain comprising the amino acid sequence of SEQ ID NO: 68; and
a heavy chain-modified hIL-2 protein fusion comprising the amino acid sequence of SEQ ID NO: 83.

18. The stable pharmaceutical formulation of claim 1, comprising 10 mg/mL of the hPD-1 antibody-modified hIL-2 immunoconjugate, 20 mM of acetate, 200 mM of sucrose, 400 mM of glycine, and 0.05% of PS80, and a pH of 5.0.

19. The stable pharmaceutical formulation of claim 1, comprising:
20 mM of acetate;
200 mM of sucrose;
400 mM of glycine;
0.05% of PS80; and
10 mg/mL of the anti-hPD-1 antibody-modified hIL-2 immunoconjugate, wherein the modified hIL-2 protein comprises the amino acid sequence of SEQ ID NO: 37 and the anti-hPD-1 antibody, or antigen-binding fragment thereof, comprises a heavy chain comprising the amino acid sequence of SEQ ID NO: 67 and a light chain comprising the amino acid sequence of SEQ ID NO: 68,
wherein the liquid pharmaceutical formulation has a pH of 5.0.

20. A stable lyophilized pharmaceutical formulation prepared by lyophilizing the stable pharmaceutical formulation of claim 1.

21. The stable lyophilized pharmaceutical formulation of claim 20, comprising:
acetate;
sucrose;
glycine;
PS80; and
an anti-hPD-1 antibody-modified hIL-2 immunoconjugate comprising:
a modified hIL-2 protein comprising the amino acid sequence of SEQ ID NO: 25; and
an anti-hPD-1 antibody, or antigen-binding fragment thereof, that immunospecifically binds to hPD-1, wherein the antibody or antigen-binding fragment thereof comprises a heavy chain CDR1 comprising the amino acid sequence of SEQ ID NO: 71, a heavy chain CDR2 comprising the amino acid sequence of SEQ ID NO: 72, a heavy chain CDR3 comprising the amino acid sequence of SEQ ID NO: 73, a light chain CDR1 comprising the amino acid sequence of SEQ ID NO: 74, a light chain CDR2 comprising the amino acid sequence of SEQ ID NO: 75, and a light chain CDR3 comprising the amino acid sequence of SEQ ID NO: 76.

22. The stable lyophilized pharmaceutical formulation of claim 21, wherein, once reconstituted, comprises 10 mg/mL of the hPD-1 antibody-modified hIL-2 immunoconjugate, 20 mM of acetate, 200 mM of sucrose, 400 mM of glycine, and 0.05% of PS80, and has a pH of 5.0.

23. A method of treating a cancer in a subject, the method comprising:
administering a therapeutically effective amount of the stable pharmaceutical formulation of claim 1 to the subject to thereby treat the cancer.

24. The method of claim 23, wherein the stable pharmaceutical formulation comprises 10 mg/mL of the hPD-1 antibody-modified hIL-2 immunoconjugate, 20 mM of acetate, 200 mM of sucrose, 400 mM of glycine, and 0.05% of PS80, and has a pH of 5.0.

25. The method of claim 23, wherein the cancer is a melanoma, a Merkel cell carcinoma, a non-small cell lung carcinoma, a renal cell carcinoma, a triple negative breast cancer, a squamous cell carcinoma of the head/neck, a hepatocellular carcinoma, or microsatellite instability-high tumors or tumors with deficient DNA mismatch repair.
